(19) United States
(12) Patent Application Publication
(10) Pub. No.: US 2025/0388480 A1
(43) Pub. Date: Dec. 25, 2025

(54) METHOD FOR SELECTING CATALYST, CATALYST, AND METHOD FOR PRODUCING CATALYST

(71) Applicant: ENEOS Corporation, Tokyo (JP)

(72) Inventors: Yoshihiro YAYAMA, Tokyo (JP); Takuya UGAJIN, Tokyo (JP)

(21) Appl. No.: 18/879,437

(22) PCT Filed: Jun. 30, 2023
(86) PCT No.: PCT/JP2023/024490

§ 371 Date: Dec. 27, 2024

(30) Foreign Application Priority Data
Jul. 4, 2022 (JP) 2022-107477

(51) Int. Cl. C01C 1/04; B01J 23/89; G16C 20/10; G16C 20/70

(52) U.S. Cl. CPC C01C 1/0411; B01J 23/8913; G16C 20/10; G16C 20/70

(57) ABSTRACT

A method for selecting a catalyst includes selecting, as a descriptor, an energy of an intermediate structure or a transition state structure included in an elementary reaction of a catalytic reaction for producing a target product from raw materials, creating a map representing a relationship between the descriptor and a reactivity of the catalyst, calculating the descriptor related to the catalytic reaction using candidate substances in a state where the candidate substances are fixed, creating a first plot map using the descriptor, selecting first screened candidate substances from the candidate substances based on the first plot map, calculating the descriptor for the catalytic reaction using the first screened candidate substances in a state where a surface of the first screened candidate substances is relaxed, creating a second plot map using the calculated descriptor, and selecting second screened candidate substances based on the second plot map.

### TECHNICAL FIELD

The present invention relates to methods for selecting catalysts, catalysts, and methods for producing catalysts.

### BACKGROUND ART

Conventionally, various compounds or the like are industrially synthesized. A target product, such as a compound or the like, is synthesized by a chemical reaction of raw materials, and a catalyst is used as a method for reducing an energy required for the chemical reaction of the raw materials.

As an example of the reaction using the catalyst for synthesizing the target product, there is a known method for synthesizing ammonia from nitrogen and hydrogen in the presence of the catalyst (for example, refer to Patent Document 1). In addition, catalysts are used for producing various compounds other than ammonia.

From a viewpoint of reducing the energy or the like for synthesizing the target product, a search for new catalysts with superior performance is ongoing.

### PRIOR ART DOCUMENTS

### Patent Documents

Patent Document 1: Japanese Laid-Open Patent Publication No. 2021-138587

### DISCLOSURE OF THE INVENTION

### Problem to be Solved by the Invention

As a method for searching catalysts, there is a method which uses first principles calculation based on the density functional theory (DFT) for calculating the energy required for synthesizing the target product from the raw materials, for example. However, in a case where the first principles calculation is used, it takes too much time to calculate the energy, and practically, it is difficult and challenging to apply the first principles calculation to a large number of (for example, several thousand) catalysts. In particular, although a transition state structure has a significant importance in catalytic reactions, the calculation takes a very long time, and it is virtually impossible to calculate the transition state structure on various catalysts. For example, in a case where 2000 kinds of transition state structures are to be calculated by the DFT-based first principles calculation, it will take several years or more to complete the calculation.

There is also a method for predicting the energy required for synthesizing the target product from the raw materials, by a general regression model using parameters of substances, such as structures, properties, or the like of the substances, such as catalysts, raw materials, or the like, as descriptors. However, although the energy can be calculated at high speed by using this prediction method, there is a problem in that a prediction accuracy is limited and an energy prediction performance is insufficient.

For this reason, because considerable time and cost are required to search for the catalyst effective for synthesizing the target product according to the methods studied so far, there are demands for a catalyst selecting method which can efficiently select the catalyst.

One object of one aspect of the present invention is to provide a method for selecting a catalyst, which can efficiently select the catalyst having a good production efficiency for a target product.

### Means of Solving the Problem

One aspect of the present invention relates to a method for selecting a catalyst to be used for a catalytic reaction for producing a target product from raw materials, the method including the steps of:
selecting, as a descriptor, an energy of an intermediate structure or a transition state structure included in an elementary reaction of the catalytic reaction;
creating a map representing a relationship between the descriptor and a reactivity of the catalyst;
preparing a plurality of kinds of candidate substances for the catalyst;
first calculating the descriptor related to the catalytic reaction using the candidate substances in a state where the candidate substances are fixed;
first plotting the descriptor calculated by the first calculating step on the map and creating a first plot map;
first screening the candidate substances based on the first plot map and selecting first screened candidate substances;
second calculating the descriptor for the catalytic reaction using the first screened candidate substances in a state where a surface of the first screened candidate substances is relaxed;
second plotting the descriptor calculated by the second calculating step on the map and creating a second plot map; and
second screening the first screened candidate substances based on the second plot map and selecting second screened candidate substances.

### Effects of the Invention

According to one aspect of the present invention, it is possible to efficiently select a catalyst having a good production efficiency for a target product.

### BRIEF DESCRIPTION OF THE DRAWINGS

FIG. 1 is a diagram illustrating a configuration of a catalyst selection system to which a method for selecting a catalyst according to an embodiment of the present invention is applied.

FIG. 2 is a diagram illustrating an example of a data table.

FIG. 3 is a diagram illustrating an example of a relationship of energy levels in each of elementary reactions in NH₃ synthesis using the catalyst.

FIG. 4 is a functional block diagram illustrating a configuration of a catalyst selection device.

FIG. 5 is a diagram illustrating an example of an activity map.

FIG. 6 is a graph illustrating an example of a relationship between an interface and a synthesis rate of NH₃ for a case where the catalyst is iron.

FIG. 7 is a functional block diagram illustrating an example of a configuration of a first calculation unit.

FIG. 8 is a diagram for explaining sorting of candidate substances in the data table.

FIG. 9 is a functional block diagram illustrating an example of a configuration of a second calculation unit.

FIG. 10 is a block diagram illustrating a hardware configuration of the catalyst selection device.

FIG. 11 is a flow chart illustrating the method for selecting the catalyst according to the embodiment of the present invention.

FIG. 12 is a diagram illustrating an example of the activity map illustrating a relationship between an adsorption energy of an intermediate of nitrogen, and a dissociation activation energy of a transition state of the nitrogen on the catalyst.

FIG. 13 is a flow chart illustrating an example of a first energy calculating step.

FIG. 14 is a diagram illustrating an example of a result of plotting the descriptors of the candidate substance.

FIG. 15 is a flow chart illustrating an example of a second energy calculating step.

FIG. 16 is a diagram illustrating an example of the activity map in which 30 kinds of second screened candidate substances are plotted.

FIG. 17 is a diagram illustrating a correlation between an energy of an intermediate or transition state of a standard catalyst calculated by DFT calculation and an energy of the intermediate or transition state of the standard catalyst, which is the same as the standard catalyst on the abscissa, calculated by the method for selecting the catalyst according to the embodiment of the present invention.

FIG. 18 is a diagram illustrating a comparison between activity maps of the catalyst selected by the catalyst selection method and the catalyst created by DFT calculation.

FIG. 19 is a flow chart illustrating an example of a method for producing the catalyst.

### MODE OF CARRYING OUT THE INVENTION

Hereinafter, embodiments of the present invention will be described in detail. In order to facilitate understanding of the description, the same constituent elements are designated by the same reference numerals in the drawings, and a redundant description of the same constituent elements will be omitted. In addition, in the present specification, when “to” is used to indicate a numerical range, the numerical range includes numerical values indicated before and after the “to” as a lower limit value and an upper limit value of the numerical range, unless indicated otherwise.

Before describing the method for selecting the catalyst according to an embodiment of the present invention, a catalyst selection system to which the method for selecting the catalyst according to the present embodiment is applied will be described. The catalyst selection system selects one or more candidates of the catalyst to be used for a catalytic reaction for producing a predetermined target product from raw materials.

In the present embodiment will be described for a case where the raw materials are hydrogen (H₂) and nitrogen (N₂), the target product is NH₃, and the catalyst is an ammonia (NH₃) synthesis catalyst.

### <Catalyst Selection System>

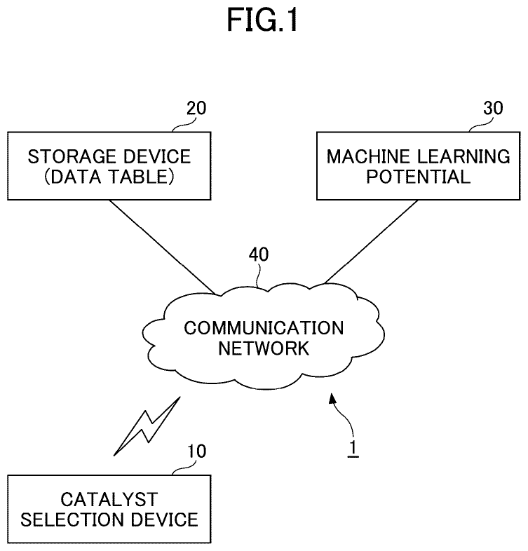

FIG. 1 is a diagram illustrating a configuration of the catalyst selection system to which the method for selecting the catalyst according to the present embodiment is applied. As illustrated in FIG. 1, a catalyst selection system 1 includes a catalyst selection device 10, a storage device 20, and a machine learning potential 30. In the catalyst selection system 1, the catalyst selection device 10, the storage device 20, and the machine learning potential 30 may be connected via a communication network 40, and input values to the catalyst selection device 10, the storage device 20, and the machine learning potential 30, and output values of the catalyst selection device 10, the storage device 20, and the machine learning potential 30 may be transmitted via the communication network 40. At least one of the storage device 20 and the machine learning potential 30 may be stored in a cloud system.

In the present embodiment, the catalyst selection device 10, the storage device 20, and the machine learning potential 30 are connected via the communication network 40, but a wired connection may be employed instead. In addition, the catalyst selection system 1 may be a single apparatus, such as a personal computer (PC) or the like, including each component within the apparatus.

The catalyst selection device 10 selects candidates for the NH₃ synthesis catalyst to be used for a catalytic reaction for synthesizing the NH₃, which is the target product, from the Ha and the N₂, which are the raw materials, using the machine learning potential 30. Details of the catalyst selection device 10 will be described later.

The storage device 20 stores a data table including information on the catalyst, an adsorbent, or the like, structures and energies of the catalyst and the adsorbent optimized using the machine learning potential 30, and a deviation or the like from a scaling line.

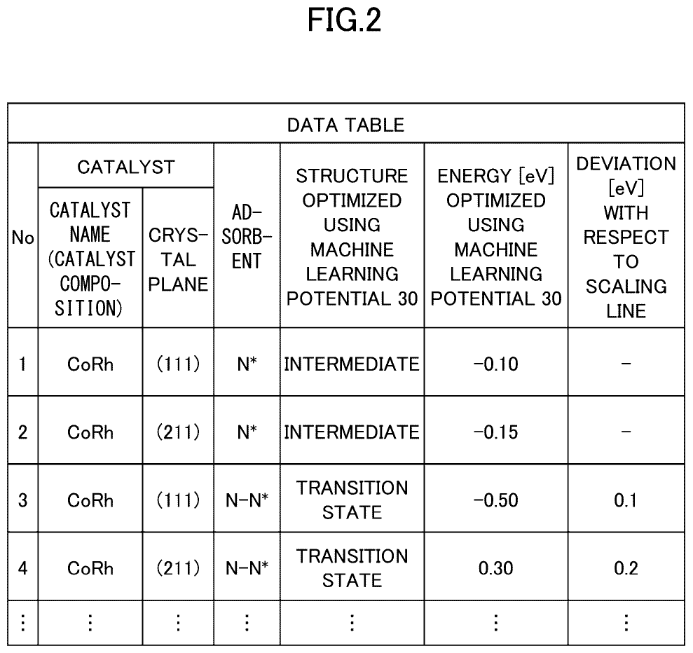

An example of the data table is illustrated in FIG. 2. As illustrated in FIG. 2, the data table records the information on the catalyst, the adsorbent, or the like, the structure and energy of the catalyst and the adsorbent optimized using the machine learning potential 30, and information on the deviation or the like from the scaling line.

The deviation from the scaling line is a difference in the dissociation activation energy of N-N* between coordinates of the energy optimized using the machine learning potential 30 (coordinates of an adsorption energy of N* and the dissociation activation energy of N-N*) and coordinates of the adsorption energy of N* on the scaling line, for the same catalyst composition and the same crystal plane. For this reason, the deviation from the scaling line is calculated only for the dissociation activation energy of N-N*.

The catalyst may be a single metal or the like, an alloy including a plurality of metals, or a compound including a metal.

Examples of the information on the catalyst include a catalyst name (catalyst composition), information on the crystal plane of the catalyst, or the like.

Examples of the catalyst name include CoRh or the like, for example.

Examples of the crystal plane of the catalyst include [001], [111], [211], or the like, for example.

The adsorbent is a component used for producing the target product, and is nitrogen (N₂) and hydrogen (H₂).

Examples of the information on the adsorbent include a name of the adsorbent or the like.

Examples of the name of the adsorbent include no adsorbent, N*, N-N*, or the like.

Examples of the optimized structures of the catalyst and adsorbent include an intermediate structure, a transition state structure, or the like of the substance.

Energies of the optimized catalyst and adsorbent include an energy of the catalytic reaction or the like. Examples of the energy of the catalytic reaction include energies of reactants that are generated in a plurality of elementary reactions in the process of synthesizing the target product NH₃ from the raw materials H₂ and N₂ in the catalytic reaction. The reactants include the raw materials, the intermediates of the raw materials, transition states of the intermediates, or the like. Examples of the energy of the reactant include a dissociation activation energy of the transition state of the intermediate of the raw materials generated by the elementary reactions in the process of synthesizing the target product from the raw materials on the catalyst, an adsorption energy of the intermediate on the catalyst, or the like.

The elementary reactions of the NH₃ synthesis include the following formulas (I) to (VII), for example. In the formulas, “*” denotes an empty adsorption site on a surface of the catalyst on which the reactant, such as an element, a molecule, or the like included in the raw materials, is adsorbed. In the formulas (I) to (VII), the raw materials are N₂ and H₂, the intermediates of the raw materials are N₂*, H*, H*, NH₂*, and NH₃*, and the transition states of the intermediates are N-N*, N—H*, NH—H*, and NH₂—H*.

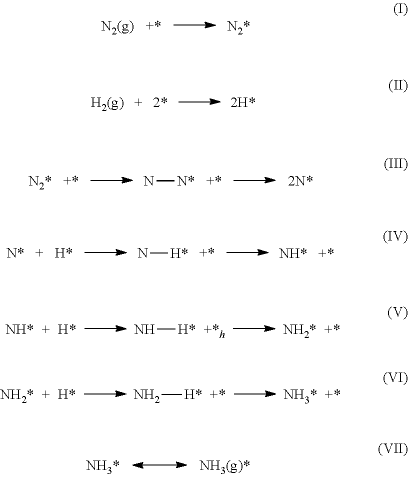

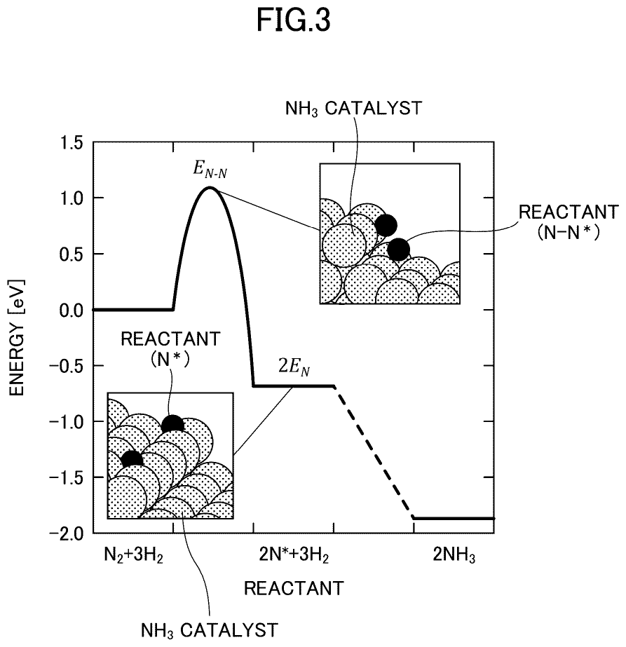

FIG. 3 illustrates an example of a relationship of energy levels in each of the elementary reactions of NH₃ synthesis using the catalysts in these formulas. As illustrated in FIG. 3, in the process of synthesizing the target product NH₃ from the raw materials H₂ and N₂, a dissociation activation energy (E_{N-N}) of N-N*, which is the transition state of nitrogen on the catalyst, is the largest, and the adsorption energy (E_{N}) of the intermediate N* of nitrogen on the catalyst is substantially constant and most stable in the formula (III) among the formulas (I) to (VII) described above. Among the formulas described above, the dissociation activation energy (E_{N-N}) of N-N* on the catalyst and the adsorption energy (E_{N}) of N* on the catalyst greatly affect the NH₃ synthesis, and can thus be suitably used as descriptors.

The machine learning potential 30 is an interatomic potential which outputs an energy from information related to an atomic structure, using a machine learning method. Examples of the machine learning potential include a neural network potential (NNP), a Gaussian approximation potential (GAP), a spectral neighbor analysis potential (SNAP), a moment tensor potential (MTP), or the like. Among these machine learning potentials, the NNP is preferable from a viewpoint of a high flexibility of the neural network. Matlantis (registered trademark) may be used for the NNP.

### [Catalyst Selection Device]

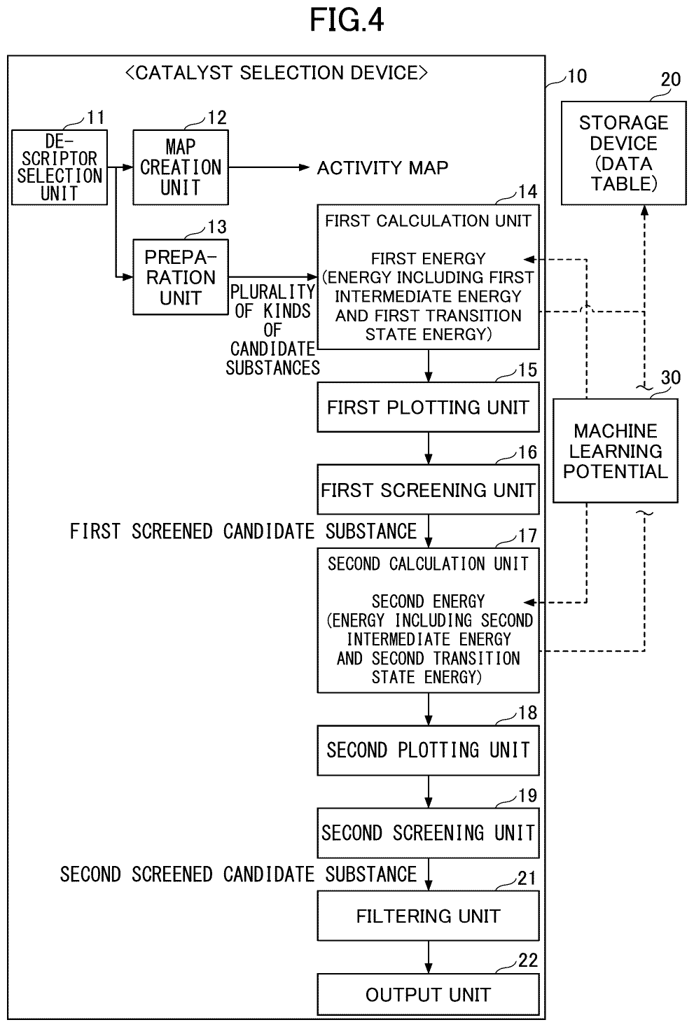

FIG. 4 is a functional block diagram illustrating a configuration of the catalyst selection device 10. As illustrated in FIG. 4, the catalyst selection device 10 includes a descriptor selection unit 11, a map creation unit 12, a preparation unit 13, a first calculation unit 14, a first plotting unit 15, a first screening unit 16, a second calculation unit 17, a second plotting unit 18, a second screening unit 19, a filtering unit 21, and an output unit 22.

The descriptor selection unit 11 selects, as the descriptor, the energy of an intermediate structure or a transition state structure included in the elementary reactions of the catalytic reaction expressed by the formulas (I) to (VII) described above.

The map creation unit 12 creates a map (activity map) representing the descriptors and a reactivity of the catalytic reaction. A map representing the descriptors and a selectivity of catalytic reaction may be created in place of the activity map.

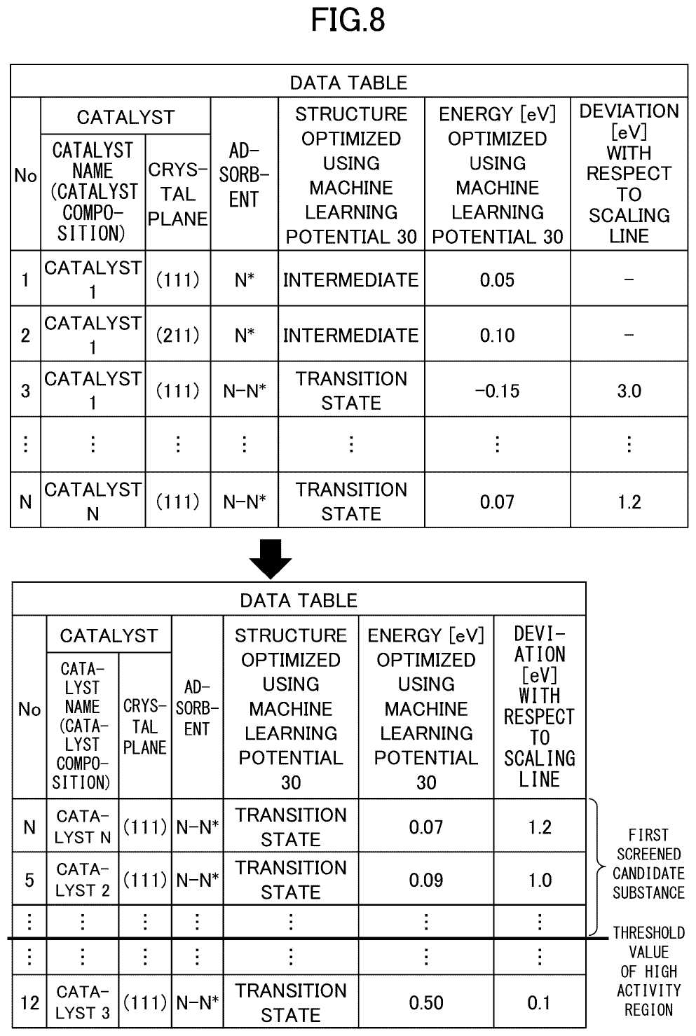

In addition, because the relationship between the descriptor and the reactivity (selectivity) of the catalyst becomes clear from the activity map, the activity map may be regarded as a model for predicting the reactivity (selectivity) of the catalyst when the descriptors are input, and the reactivity of the catalyst may be predicted directly from the descriptors. By regarding the activity map as a model, the reactivity of the catalyst may be predicted directly from the descriptors obtained by the first calculation unit 14 and the second calculation unit 17, and the candidate substance may be screened according to the reactivity using a data table (refer to FIG. 8). In this case, the catalyst selection device 10 does not require the first plotting unit 15 and the second plotting unit 18.

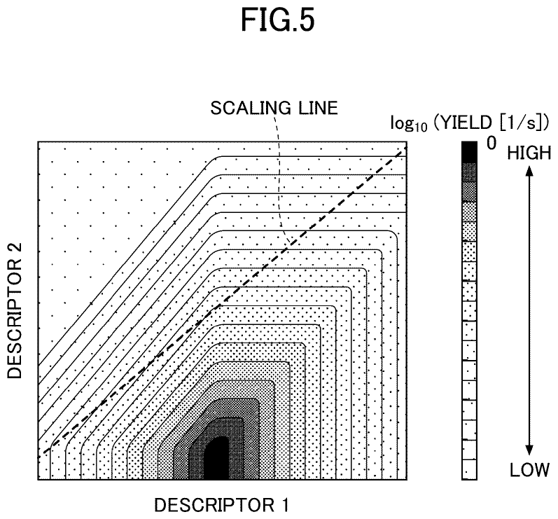

An example of the activity map is illustrated in FIG. 5. FIG. 5 represents a relationship between two descriptors of the elementary reactions of the reactants in the NHs synthesis (refer to the formulas (I) to (VII) described above) and a yield of the synthesized NH₃. The two descriptors used in the activity map illustrated in FIG. 5 will be referred to as descriptors 1 and 2. As described above, among the formulas (I) to (VII), the dissociation activation energy (E_{N-N}) of the transition state (N-N*) of the intermediate of the nitrogen on the catalyst, and the adsorption energy (EN) of the intermediate (N*) of nitrogen on the catalyst greatly affect the NH₃ synthesis. For this reason, the descriptors 1 and 2 preferably use the adsorption energy (E_{N}) of N* on the catalyst and the dissociation activation energy (E_{N-N}) of N-N* on the catalyst.

The activity map can be created by microkinetics using the descriptors, for example. In the present embodiment, because the catalyst is the NH₃ synthesis catalyst, and a NH₃ synthesis reaction is generated, it is preferable, as described above, to use the energies of N* and N-N* in the formulas of the elementary reactions (refer to the formulas (I) to (VII) described above) of the reactants in the NH₃ synthesis, as the descriptors. When the energies of N* and N-N* are used as the descriptors, a linear relationship among a plurality of catalysts can be expressed based on levels of reaction rates of the catalysts, and thus, it is possible to obtain a map in which a region having a high activity can be visually understood with ease based on the levels of the reaction rates. A straight line indicated on the activity map can be used as a scaling line (refer to FIG. 5) which serves as a threshold value of the reaction rates of the catalysts, as will be described later.

A general microkinetics will be described, before describing the microkinetics using the descriptors. The general microkinetics include the following enumeration of elementary reactions, energy calculation, and reaction rate calculation.

### 1. Enumeration of Elementary Reactions:

All elementary reactions related to the NH₃ synthesis are enumerated.

### 2. Energy Calculation:

The energy of all of the intermediates and transition states included in the enumerated elementary reactions, on the surface of a specific catalyst (for example, the (111) plane of the catalyst) are calculated by the machine learning potential 30.

### 3. Reaction Rate Calculation:

The calculated energy is converted into a reaction rate formula, and the reaction rate of each elementary reaction obtained in the “1. Enumeration of Elementary Reactions:” described above is obtained by solving simultaneous ordinary differential equations. Then, the rate of “NH₃*<=>NH₃(g)*” in the formula (VII) of the elementary reactions related to the NH₃ synthesis is output, so as to obtain a NH₃ synthesis rate on the surface of the catalyst (for example, the (111) plane of the catalyst).

Next, the microkinetics using the descriptors will be described. A procedure for performing the microkinetics using the descriptors is as follows.

### 1. Preparation:

1-1. All elementary reactions related to the NH₃ synthesis are described.

1-2. All intermediates and transition states generated in the elementary reactions related to the NH₃ synthesis are calculated. For example, for some standard catalysts, all intermediates and transition states are calculated. That is, all intermediates and transition states that are generated when these standard catalysts are used are calculated. Then, only two reactants (for example, the intermediate or the transition state) used for the descriptors are selected from all of the intermediates and transition states, and two descriptors (for example, the adsorption energy of N* and the dissociation activation energy of N-N*) are calculated. Other parameters (for example, the energy of the intermediate, the energy of the transition state) are acquired by a linear regression from the descriptors.

### 2. Creating Activity Map:

When the values of the two descriptors are determined, the energies of all of the intermediates and transition states included in all of the enumerated elementary reactions can be obtained from the linear regression, and the NH₃ synthesis rate can be obtained by the general microkinetics of the “3. Reaction Rate Calculation:” of the microkinetics described above. By calculating the NH₃ synthesis rate while varying the values of the two descriptors in an arbitrary range, the activity map as illustrated in FIG. 5 can be obtained.

### 3. New Catalyst Screening:

With respect to new catalysts, because the relationship between the energies of the two descriptors and the NH: synthesis rate is already clarified in the “2. Creating Activity Map:” described above, the synthesis rate of the catalyst can be acquired by calculating only the two descriptors (for example, the adsorption energy of N* and the dissociation activation energy of N-N*).

Because the energies of all of the intermediates and transition states included in the elementary reactions need to be calculated as described above in order to perform the general microkinetics, it is necessary to calculate an extremely large number of parameters. In contrast, when the microkinetics is performed using the descriptors, the energies of all of the intermediates and transition states in the elementary reactions for a standard catalyst are calculated in advance, and thus, only calculations corresponding to two descriptors are required even for an unknown catalyst. When the two descriptors are plotted on an X-axis and a Y-axis to create the activity map, the NH₃ synthesis catalyst belonging to a region where the reaction rates of the catalyst are high (a high activity region which will be described later) can be intuitively understood from the activity map.

The number of descriptors is not limited to two, and may be one or three or more, and descriptors which affect the reaction rate of the catalyst are preferably used. Further, for the calculation of the other parameters, not only the linear regression but also a non-linear regression method or the like may be used.

As illustrated in FIG. 4, the preparation unit 13 prepares a plurality of kinds of candidate substances for the catalyst.

The candidate substances for the catalyst may be a material used in the NH₃ synthesis, a material being considered for use in the NH₃ synthesis, a material never used in the NH; synthesis, or the like. The material may be an elemental metal, or an alloy including a plurality of metals, or a metal compound such as an oxide, a nitride, a carbide, or the like of a metal. The candidate substance may include a substance in which one kind of element on a surface of one candidate substance is substituted with another different element, as another candidate substance different from the one candidate substance.

The number of candidate substances for the catalyst is not particularly limited and can be selected, as appropriate, and may be several tens, several hundreds, several thousands, or the like, for example.

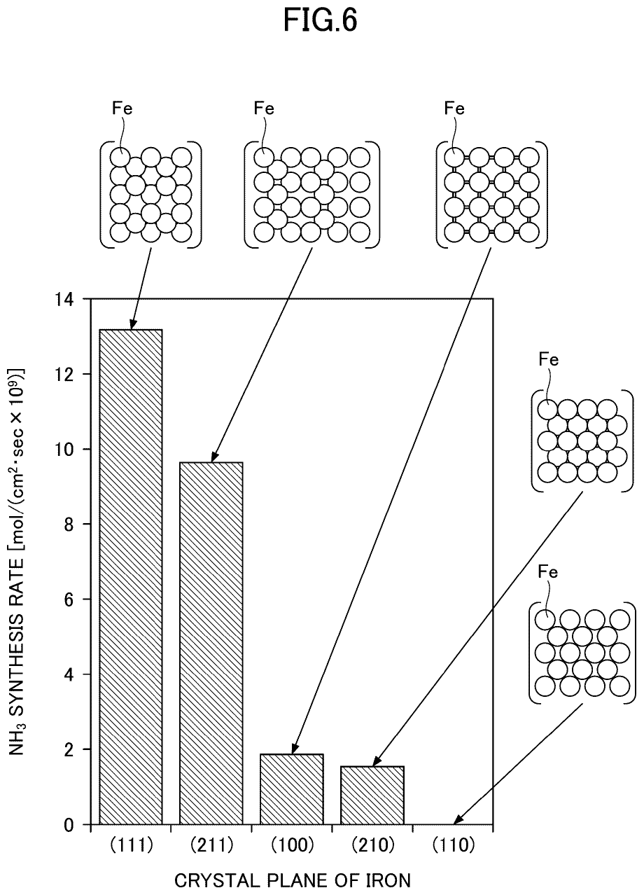

In a case where the candidate substance has a plurality of surfaces having different crystal planes, a plurality of candidate substances may be prepared for each of the surfaces having the different crystal planes. The reaction rate between the candidate substance and the element tends to differ depending on the crystal plane of the candidate substance appearing at the surface of the candidate substance. FIG. 6 illustrates an example of a relationship between the crystal plane of iron (Fe) and the NH₃ synthesis rate, for a case where the catalyst is iron, for example. As illustrated in FIG. 6, in the case where the catalyst is iron, the NH₃ synthesis rate differs depending on the crystal plane at the surface of the catalyst, and in a case where the crystal plane is the (111) plane or the (211) plane, the catalyst has a higher NH₃ synthesis rate compared to cases where other crystal planes are present at the surface of the catalyst. For this reason, it is preferable to prepare a plurality of surfaces of the candidate substance for each of the crystal planes.

As illustrated in FIG. 4, the first calculation unit 14 calculates the descriptors related to the catalytic reaction using the candidate substance, in a state where all of the candidate substances prepared by the preparation unit 13 are fixed.

The present embodiment calculates, as the descriptor, a first energy including a first intermediate energy of an intermediate of a reactant including a substance derived from raw materials (N₂, H₂) and adsorbed on a surface of a candidate substance, and a first transition state energy of a dissociation reaction in which the reactant is separated into two or more substances after a transition state of the intermediate of the reactant is adsorbed on the surface of the candidate substance.

The state where all of the candidate substances are fixed refers to a state in which positions and crystal structures of the candidate substances are fixed by assuming that that the positions and the crystal structures of the candidate substances do not move when the candidate substances react with the N₂ and the H₂.

Further, the first calculation unit 14 may calculate only the first intermediate energy or the first transition state energy as the first energy.

The first calculation unit 14 preferably calculates the descriptor using the machine learning potential 30. By using the machine learning potential 30, the first calculation unit 14 can reduce a calculation time of the descriptor compared to a case where first principles calculation (DFT calculation) or the like based on the density functional theory (DFT) is used.

The first calculation unit 14 may determine the descriptor by one calculation or may determine the descriptor by a plurality of calculations (for example, 10 calculations). In the case where the calculation is performed a plurality of times, an average value of a plurality of calculated values, or a maximum value or a minimum value of the plurality of calculated values may be used for the descriptor.

In the case where the descriptor is the first intermediate energy, the first calculation unit 14 preferably varies the adsorption position of the reactant including the substance derived from the raw materials on the surface of the candidate substance included in the elementary reaction, in the state where all of the candidate substances are fixed, and optimizes the structure of the intermediate structure including the candidate substance and the reactant. In this case, the first calculation unit 14 can select the intermediate structure having a stable structure.

Optimization of the structure refers to obtaining an optimum value that minimizes the energy of a predetermined structure. The same applies to the optimization of the structure in the following description.

The first calculation unit 14 preferably extracts a structure in which the adsorption position of the reactant with respect to the candidate substance is optimal and the first intermediate energy of the candidate substance and the intermediate of the reactant is most stable. In this case, the first calculation unit 14 can acquire the first intermediate energy of the intermediate of the reactant in a short time, although with a low accuracy.

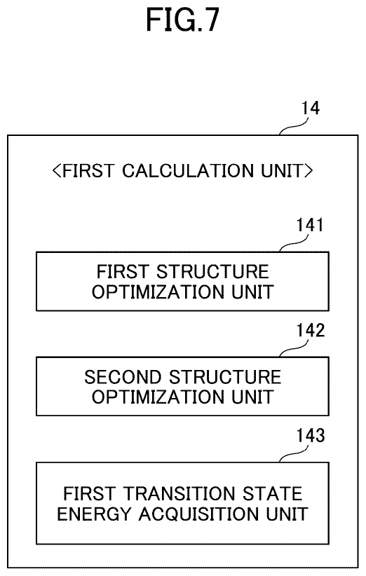

In addition, as illustrated in FIG. 7, the first calculation unit 14 preferably includes a first structure optimization unit 141, a second structure optimization unit 142, and a first transition state energy acquisition unit 143. In the case where the descriptor is the transition state energy of the transition state of the intermediate, the first calculation unit 14 can acquire the first transition state energy in a short time, although with a low accuracy.

The first structure optimization unit 141 optimizes the structure of an intermediate structure including a candidate substance and a reactant of the reactant on a surface of the candidate substance included in a dissociation reaction from one molecule to two molecules, and calculates a first optimized structure. That is, the first structure optimization unit 141 calculates the first optimized structure by optimizing the structure of the intermediate structure including the candidate substance and the reactant, in a state where all of the structures of the candidate substance are fixed, in the dissociation reaction from one molecule to two molecules.

The second structure optimization unit 142 optimizes the structure when the reactant is separated into two or more substances including the candidate substance and the reactant, and calculates a second optimized structure.

The first transition state energy acquisition unit 143 calculates a first transition state energy, which is a transition state energy, from the first optimized structure obtained by the first structure optimization unit 141 and the second optimized structure obtained by the second structure optimization unit 142.

For example, assuming that AB* is a molecule in a state adsorbed on the candidate substance, A-B* is a molecule in a transition state of AB* in a process of separating AB* into A* and B*, A* is an intermediate of a molecule A, and B* is an intermediate of a molecule B, a reaction in which one molecule AB* separates into two molecules A* and B* progresses as indicated by the following reaction formula (1).

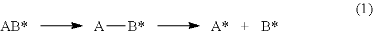

In a case where a dissociation reaction in which AB* separates from one molecule into two molecules occurs as indicated by the formula (1) described above, the first structure optimization unit 141 arranges AB* on the surface of the candidate substance in a state where all of the structures of the candidate substance are fixed, and optimizes the structure of the intermediate structure including the candidate substance and AB*.

The second structure optimization unit 142 optimizes the structure so that the structure separates into two molecules A* and B*.

The first transition state energy acquisition unit 143 calculates, as the first transition state energy, a transition state energy required for one molecule AB* to separate into two molecules A* and B*, using the nudged elastic band (NEB) method.

As illustrated in FIG. 4, the first plotting unit 15 plots the candidate substance based on the first energy (the energy including the first intermediate energy and the first transition state energy), which is the descriptor calculated by the first calculation unit 14, on the activity map (refer to FIG. 5) displayed by the map creation unit 12, and creates a first plot map. The first screening unit 16 screens the candidate substances based on the first plot map created by the first plotting unit 15, and selects first screened candidate substances. That is, the first screening unit 16 employs a screening method which narrows down the candidate substances to candidate substances plotted in a high activity region including catalysts having a high activity (a high NH₃ synthesis rate), as the first screened candidate substances.

The high activity region may be a range smaller by a predetermined value or more with respect to the scaling line, by providing the scaling line on the activity map plotted with the candidate substances (refer to FIG. 5). In addition, the candidate substances may be narrowed down to a range less than a NH₃ producing energy of the catalyst by a predetermined value. The range may be a range in which the NH₃ synthesis rate is greater than or equal to a predetermined value. The predetermined value of the synthesis rate is preferably 10⁻⁴ [l/s] or greater.

The number of candidate substances to be further narrowed down can be appropriately selected according to the range of the high activity region to be set, and is preferably 5 to 30, more preferably 8 to 55, and still more preferably 10 to 20, for example.

The activity map can be created by the microkinetics using the descriptors, as described above. By using the energies of N* and N-N* in the formulas of the elementary reaction (refer to the formulas (I) to (VII) described above) of the reactant in the NH₃ synthesis as the descriptors, the activity map can be expressed as having a linear relationship for the descriptors of the standard catalyst, such as elemental metals such as Co, Rh, Ru, Cu, Fe, Re, or the like which will be described later. A straight line of this linear relationship can be used as the scaling line (refer to FIG. 5).

The producing energy can be calculated according to the following formula (i). In a case where the producing energy is less than 0 (the producing energy <0), it indicates that the alloy is more stable than the element metal.

Producing Energy=E_{Alloy}−N_{A}×E_{A(bulk)}−N_{B}×N_{B(bulk)} (i)

(In the formula, E_{Alloy} denotes a bulk energy of the alloy AB, E_{A(bulk)} denotes a bulk energy of a metal A, E_{B(bulk)} denotes a bulk energy of a metal B, N_{A} denotes an atomic number of the metal A in the alloy, and N_{B} denotes an atomic number of the metal B in the alloy.)

The range smaller by the predetermined value or more with respect to the scaling line can be appropriately selected according to the target product to be synthesized, a type of the material to be used, or the like, but in the case of the NH₃ synthesis catalyst, the range is preferably smaller by 0.25 eV or more with respect to the scaling line, for example.

The range less than the producing energy by the predetermined value can be appropriately selected according on the target product to be synthesized, the type of the material to be used, or the like, but in the case of the NH₃ synthesis catalyst, the range is preferably less than the producing energy by 0.05 eV/atom, for example.

A number of kinds of first screened candidate substances to be narrowed down to can be appropriately selected according to the number of candidate substances, a number of second screened candidate substances which will be described later, or the like, and may be several tens of kinds or several hundreds of kinds, for example.

In addition, when the first screening unit 16 narrows down the first screened candidate substances by plotting the candidate substances on the activity map, the first screening unit 16 may use a data table in which only information related to candidate substances 1, 2, 3, . . . , N (N is an integer greater than or equal to 1) is listed, as illustrated in FIG. 8. The candidate substances in the data table listing only the candidate substances may be sorted in an order included in the high activity region, and the candidate substances included in the high activity region and greater than or equal to a threshold value may be narrowed down as the first screened candidate substances. The first screening unit 16 may narrow down the first screened candidate substances by plotting the candidate substances on the activity map by the first plotting unit 15, using the sorted data table.

As illustrated in FIG. 4, the second calculation unit 17 calculates, with respect to the first screened candidate substances, the descriptors related to catalytic reactions using the first screened candidate substances, in a state where the surface of the first screened candidate substances is relaxed.

The present embodiment calculates, as the descriptor, a second energy including a second intermediate energy of an intermediate of the reactant (N₂, H₂) used for the descriptor and adsorbed on the surface of the first screened candidate substances, and a second transition state energy of a dissociation reaction in which the reactant is separated into two or more substances after the reactant is adsorbed on the surface of the first screened candidate substances, similar to the first energy.

The state where the surface of the catalyst is relaxed refers to a state in which the catalyst is allowed to move, such that the structure can vary by moving several layers (for example, approximately two layers) from the surface of the catalyst, and is a state close to an actual movement of the catalyst.

In addition, the second calculation unit 17 may calculate only the second intermediate energy or the second transition state energy as the second energy.

The second calculation unit 17 preferably calculates the descriptor using the machine learning potential 30, similar to the first calculation unit 14. By using the machine learning potential 30, the second calculation unit 17 can reduce the calculation time of the descriptor compared to the case where the DET calculation or the like is used.

In the case where the descriptor is the second intermediate energy, the second calculation unit 17 preferably varies the adsorption position of the reactant including the substance derived from the raw materials on the surface of the first screened candidate substances included in the elementary reaction, similar to the first calculation unit 14, in the state where the surface of the first screened candidate substances is relaxed, and optimizes the structure of the intermediate structure including the first screened candidate substance and the reactant. In this case, the second calculation unit 17 can select the intermediate structure having a stable structure.

Similar to the first calculation unit 14, the second calculation unit 17 preferably extracts a structure in which the adsorption structure of the reactant with respect to the first screened candidate substances is correct, and the second intermediate energy of the first screened candidate substances and the intermediate of the reactant is most stable. By optimizing the structure, the structure may vary from the structure of the molecule (for example, the structure of AB*) in the state adsorbed on the candidate substance, similar to the case where the structure is optimized in the first structure optimization unit 141. The second calculation unit 17 can exclude such a structure which varies, and can thus acquire the second intermediate energy of the reactant with a high accuracy.

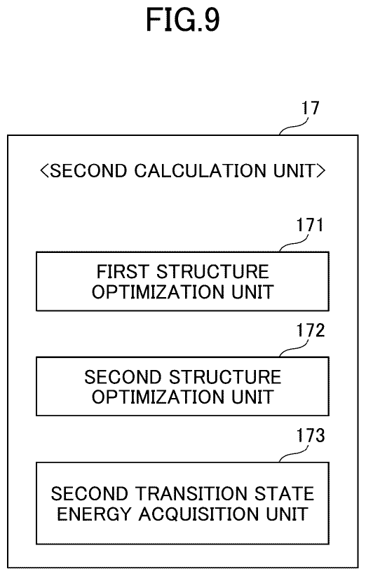

Further, the second calculation unit 17 preferably includes a first structure optimization unit 171, a second structure optimization unit 172, and a second transition state energy acquisition unit 173, as illustrated in FIG. 9. In this case, the second calculation unit 17 can acquire the second transition state energy with a high accuracy in the case where the descriptor is the transition state energy of the transition state of the intermediate.

The first structure optimization unit 171 optimizes the structure of the intermediate structure including the first screened candidate substance and the reactant on the surface of the first screened candidate substances included in the dissociation reaction from one molecule to two molecules, and calculates the first optimized structure. That is, the first structure optimization unit 171 optimizes the structure of the intermediate structure including the first screened candidate substance and the reactant in the state where the surface of the first screened candidate substances is relaxed in the dissociation reaction from one molecule to two molecules, and calculates the first optimized structure.

The second structure optimization unit 172 optimizes a separation structure when the reactant is separated into two or more substances, and calculates the second optimized structure, similar to the second structure optimization unit 142.

The second transition state energy acquisition unit 173 calculates the second transition state energy, which is the transition state energy, from the first optimized structure obtained by the first structure optimization unit 171 and the second optimized structure obtained by the second structure optimization unit 172, similar to the first transition state energy acquisition unit 143.

For example, as described above, AB* is the molecule in the state adsorbed on the candidate substance, A-B* is the molecule in the transition state of AB* in the process of separating AB* into A* and B*, A* is the intermediate of the molecule A, B* is the intermediate of the molecule B, and the reaction in which one molecule AB* separates into two molecules A* and B* progresses as indicated by the reaction formula (1) described above.

In the dissociation reaction in which AB* separates from one molecule into two molecules as indicated by the formula (1) described above, the first structure optimization unit 171 arranges AB* on the surface of the first screened candidate substances in the state where the surface of the first screened candidate substances is relaxed, and optimizes the structure of the intermediate structure including the first screened candidate substance and AB*, similar to the first structure optimization unit 141.

The second structure optimization unit 172 optimizes the separation structure so that the separation structure separates into two molecules A* and B*.

The second transition state energy acquisition unit 173 calculates, as the second transition state energy, the transition state energy required for one molecule AB* to separate into two molecules A* and B*, similar to the first transition state energy acquisition unit 143.

As illustrated in FIG. 4, the second plotting unit 18 plots the first screened candidate substances on the activity map (refer to FIG. 5) based on the second energy which is the descriptor calculated by the second calculation unit 17, and creates a second plot map.

The second screening unit 19 screens the first screened candidate substances based on the second plot map created by the second plotting unit 18, and selects second screened candidate substances. That is, the second screening unit 19 can select the first screened candidate substances plotted in the high activity region of the second plot map as the second screened candidate substances, using the screening method.

The method of screening the first screened candidate substances is the same as the method of narrowing down the candidate substances to the first screened candidate substances by the first screening unit 16, and thus, details thereof will be omitted.

A number of kinds of second screened candidate substances to be selected can be appropriately selected according to the number of the first screened candidate substances or the like, and may be several tens of kinds or several hundreds of kinds, for example.

The filtering unit 21 narrows down the second screened candidate substances selected by the second screening unit 19, based on a catalyst stability, a catalyst cost, or the like.

The number of kinds of second screened candidate substances to be narrowed down to can be appropriately selected according to the number of second screened candidate substances to be selected or the like, and may be several kinds or several tens of kinds, for example.

The output unit 22 outputs the second screened candidate substance selected by the second screening unit 19 or the second screened candidate substances narrowed down by the filtering unit 21, to a display or the like.

In the present embodiment, the catalyst selection device 10 does not require the first calculation unit 14, the first plotting unit 15, and the first screening unit 16, or the second calculation unit 17, the second plotting unit 18, and the second screening unit 19, according to the number of candidate substances in the preparation unit 13. That is, the catalyst selection device 10 may narrow down the candidate substances by omitting the calculation in the state where all of the candidate substances are fixed or by omitting the calculation in the state where the surface of the candidate substances is relaxed.

### (Hardware Configuration of Catalyst Selection Device 10)

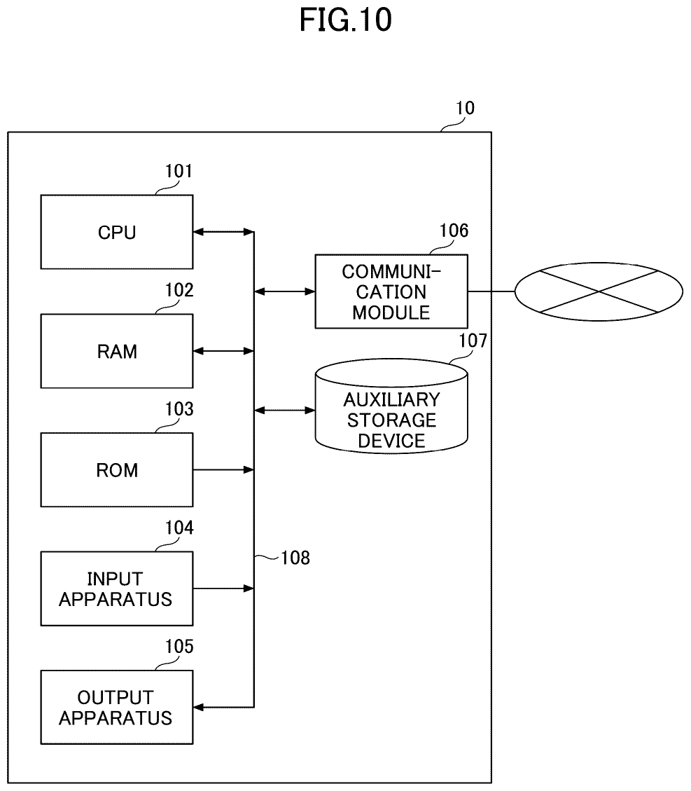

Next, an example of a hardware configuration of the catalyst selection device 10 will be described. FIG. 10 is a block diagram illustrating the hardware configuration of the catalyst selection device 10. As illustrated in FIG. 10, the catalyst selection device 10 is configured by an information processing apparatus (computer), and can be physically configured as a computer system including a central processing unit (CPU: processor) 101 as an arithmetic processing unit, a random access memory (RAM) 102 and a read only memory (ROM) 103 as a main storage device, an input apparatus 104 as an input device, an output apparatus 105, a communication module 106, an auxiliary storage device 107 such as a hard disk or the like, or the like. These components are connected to each other via a bus 108. The output apparatus 105 and the auxiliary storage device 107 may be provided externally.

The CPU 101 controls an overall operation of the catalyst selection device 10, and performs various information processing. The CPU 101 can select a catalyst by executing a catalyst selection method or a catalyst selection program which will be described later and is stored in the ROM 103 or the auxiliary storage device 107.

The RAM 102 may include a non-volatile RAM which is used as a work area of the CPU 101 and stores main control parameters and information.

The ROM 103 stores a basic input/output system or the like. The catalyst selection program may be stored in the ROM 103.

The input apparatus 104 is an input device, such as a keyboard, a mouse, an operation button, a touchscreen panel, a display screen, or the like, and receives information input by a user as an instruction signal and outputs the instruction signal to the CPU 101.

The output apparatus 105 is a display device such as a monitor display or the like, a speaker, a printing device such as a printer or the like, or the like. In the output apparatus 105, information on a catalyst selection result or the like is displayed on the display device such as the monitor display or the like, for example, and a display screen is updated in response to an input operation via the input apparatus 104 or the communication module 106.

The communication module 106 is a data transmission and reception device, such as a network card or the like, and functions as a communication interface which receives information from an external data recording server or the like, and outputs analysis information to another electronic device.

The auxiliary storage device 107 is a storage device, such as a solid state drive (SSD), a hard disk drive (HDD), or the like, and stores various data, files, or the like required for the operation of the catalyst selection device 10, for example.

The functions of the catalyst selection device 10 are implemented by reading predetermined computer software (including the catalyst selection program) from the main storage device such as the RAM 102 or the like, or from the auxiliary storage device 107, and executing the software by the CPU 101, thereby reading data from and writing data to the main storage device such as the RAM 102 or the like, or the auxiliary storage device 107, and operating the input apparatus 104, the output apparatus 105, and the communication module 106.

Accordingly, each component of the catalyst selection device 10 illustrated in FIG. 4 can be implemented by cooperation of software and hardware, by executing the prestored predetermined computer software (including the catalyst selection program) by the processor in the computer including the catalyst selection device 10.

The catalyst selection program may be stored in the main storage device or the auxiliary storage device 107 included in the computer, for example. The catalyst selection program may be stored in a computer connected to a communication line, such as the Internet or the like, and the catalyst selection program may be provided by downloading a part or all of the catalyst selection program via the communication line. Further, the catalyst selection program may be provided or distributed via the communication line.

The catalyst selection program may be recorded (including being installed) in the computer from a state where a part or all of the catalyst selection program is stored in a portable storage medium, such as an optical disk such as a CD-ROM, a DVD-ROM, or the like, a semiconductor memory such as a flash memory or the like, or the like.

### <Method for Selecting Catalyst>

A method for selecting a catalyst according to the present embodiment will be described. The method for selecting the catalyst according to the present embodiment can be performed using the catalyst selection system 1 described above. For this reason, a description of the items already described above will be omitted in part.

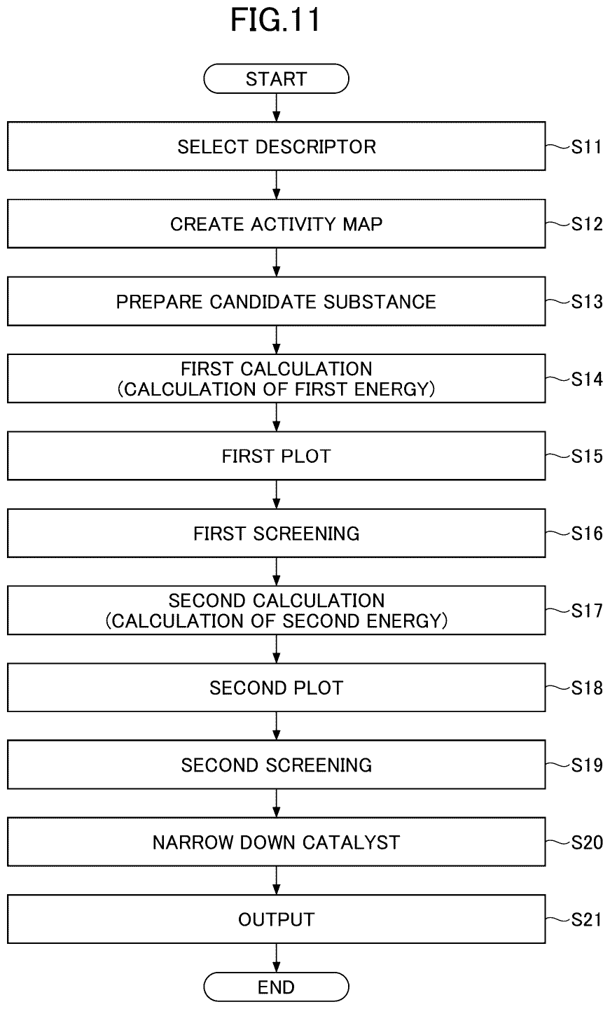

FIG. 11 is a flow chart illustrating the method for selecting the catalyst according to the present embodiment. As illustrated in FIG. 11, the method for selecting the catalyst according to the present embodiment is a method for selecting the NH₃ synthesis catalyst to be used for the catalytic reaction for producing NH₃, which is the target product, from H₂ and N₂ which are the raw materials.

In the method for selecting the catalyst according to the present embodiment, the descriptor selection unit 11 selects, as the descriptor, the energy of the intermediate structure or the transition state structure included in the elementary reactions of the catalytic reaction expressed by the formulas (I) to (VII) described above (descriptor selection process: step S11).

As the descriptor of the catalytic reaction, it is preferable to use the adsorption energy of the reactant adsorbed on the surface of the candidate substance, and the transition state energy of the dissociation reaction in which the reactant is separated into two or more substances after the reactant is adsorbed on the surface of the candidate substance. For example, the adsorption energy (E_{N}) of the intermediate of nitrogen (N*) on the catalyst is used as the adsorption energy of the reactant adsorbed on the surface of the candidate substance. The dissociation activation energy (E_{N-N}) of the transition state (N-N*) of nitrogen on the catalyst is used as the transition state energy of the dissociation reaction in which the reactant is separated into two or more substances after the reactant is adsorbed on the surface of the candidate substance.

Next, the map creation unit 12 creates the activity map representing the descriptors and the reactivity of the catalytic reaction (activity map creation process: step S12).

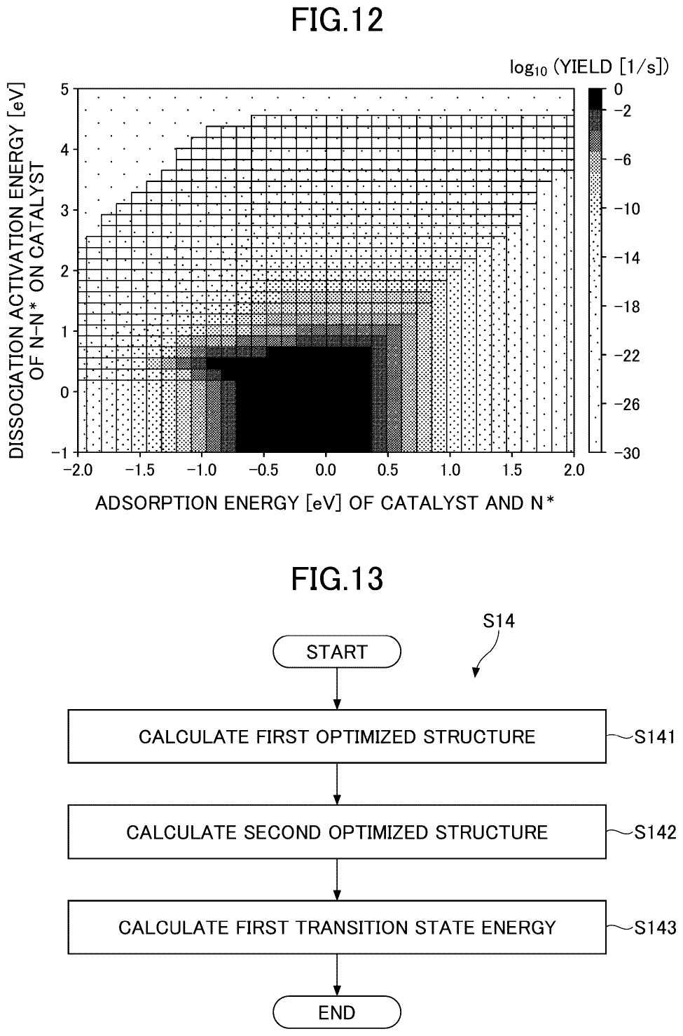

FIG. 12 illustrates an example of the activity map for a case where the descriptors of the catalytic reaction are the adsorption energy (E_{N}) of the intermediate (N*) of nitrogen, and the dissociation activation energy (E_{N-N}) of the transition state (N-N*) of nitrogen on the catalyst.

Next, the preparation unit 13 prepares several thousand kinds (for example, 2000 kinds) of candidate substances for the catalyst (preparation process: step S13).

Although the number of kinds of candidate substances to be prepared is several thousand in this example, the number of kinds of candidate substances is not limited to such, and may be an arbitrary number, as appropriate.

Next, the first calculation unit 14 calculates the descriptors related to the catalytic reactions using the candidate substances in a state where all of the candidate substances prepared by the preparation unit 13 are fixed (first calculation process: step S14).

As described above, the present embodiment calculates, as the descriptor, the first energy including the first intermediate energy of the intermediate of the reactant including the substance derived from the raw materials (N₂, H₂) and adsorbed on the surface of the candidate substance, and the first transition state energy of the dissociation reaction in which the reactant is separated into two or more substances after the transition state of the intermediate of the reactant is adsorbed on the surface of the candidate substance.

In the first calculation process (step S14), the first calculation unit 14 preferably calculates the descriptor of the candidate substance, using the machine learning potential 30.

The first energy may be determined by one calculation or may be determined by a plurality of calculations (for example, 10 calculations). In the case where the calculation is performed a plurality of times, an average value of a plurality of calculated values, or a maximum value or a minimum value of the plurality of calculated values may be used for the first energy.

In the first calculation process (step S14), in the case where the descriptor is the first intermediate energy, the first calculation unit 14 preferably varies the adsorption position of the reactant including the substance derived from the raw materials on the surface of the candidate substance included in the elementary reaction, in the state where all of the candidate substances are fixed, and optimizes the structure of the intermediate structure including the candidate substance and the reactant. In this case, the intermediate structure having a stable structure can be selected.

In the first calculation process (step S14), the first calculation unit 14 preferably extracts the structure in which the adsorption structure of the reactant with respect to the candidate substance is correct, and the first intermediate energy of the candidate substance and the intermediate of the reactant is most stable. In this case, the first intermediate energy of the candidate substance and the intermediate of the reactant can be calculated in a short time, although with a low accuracy.

In addition, the first calculation process (step S14) preferably includes a first structure optimization process (step S141), a second structure optimization process (step S142), and a first transition state energy acquisition process (step S143), as illustrated in FIG. 13. In this case, the first transition state energy of the transition state of the intermediate can be calculated as the descriptor in a short time, although with a low accuracy.

In the first structure optimization process (step S141), the first structure optimization unit 141 optimizes the structure of the intermediate structure including the candidate substance of the reactant and the reactant on the surface of the candidate substance included in the dissociation reaction from one molecule to two molecules, and calculates the first optimized structure. That is, in the first structure optimization process (step S141), the first structure optimization unit 141 optimizes the structure of the intermediate structure including the candidate substance and the reactant, in the state where all of the structures of the candidate substance are fixed, in the dissociation reaction from one molecule to two molecules, and calculates the first optimized structure.

The second structure optimization process (step S142) optimizes the structure when the reactant is separated into two or more substances including the candidate substance of the reactant and the reactant, and calculates the second optimized structure.

The first transition state energy acquisition process (step S143) calculates the first transition state energy, which is the transition state energy, from the first optimized structure obtained in the first structure optimization process (step S141) and the second optimized structure obtained in the second structure optimization process (step S142).

For example, AB* is the molecule adsorbed on the candidate substance, A-B* is the molecule in the transition state of AB* in the process of separating AB* into A* and B*, A* is the intermediate of the molecule A, B* is the intermediate of the molecule B, as described above, and the reaction in which one molecule AB* separates into two molecules A* and B* is assumed to progress as in the reaction formula (1) described above.

As indicated by the formula (1) described above, in the case where the dissociation reaction in which AB* changes from one molecule to two molecules occurs, the first structure optimization process (step S141), arranges by the first structure optimization unit 141, AB* on the surface of the candidate substance in the state where all of the structures of the candidate substances are fixed, and optimizes the structure of the intermediate structure including the candidate substance and AB*.

The second structure optimization process (step S142) optimizes the structure so that AB* separates into two molecules A* and B*.

In the first transition state energy acquisition process (step S143), the first transition state energy acquisition unit 143 calculates, as the first transition state energy, the transition state energy required for one molecule AB* to separate into two molecules A* and B*, using the nudged elastic band (NEB) method.

Next, as illustrated in FIG. 11, the first plotting unit 15 plots the candidate substances on the activity map (refer to FIG. 12) created in the map creation process (step S12) based on the first energy (the energy including the first intermediate energy and the first transition state energy) which is the descriptor calculated in the first calculation process (step S14), and creates a first plot map (first plotting process: step S15).

Next, the first screening unit 16 screens the candidate substances based on the first plot map created in the first plotting process (step S15), and selects the first screened candidate substances (first screening process: step S16).

That is, the first screening unit 16 narrows down the candidate substances plotted in the high activity region including the catalyst having a high activity (high NH₃ synthesis rate) as the first screened candidate substances.

The method and meaning of the setting the high activity region are as described above, and thus, a description of the details thereof will be omitted.

The first screened candidate substances are narrowed down to 100 kinds to 500 kinds, for example. The number of first screened candidate substances to be narrowed down to can be appropriately selected as described above according to the number of candidate substances, the number of second screened candidate substances, or the like, and may be several tens of kinds or several hundreds of kinds, for example.

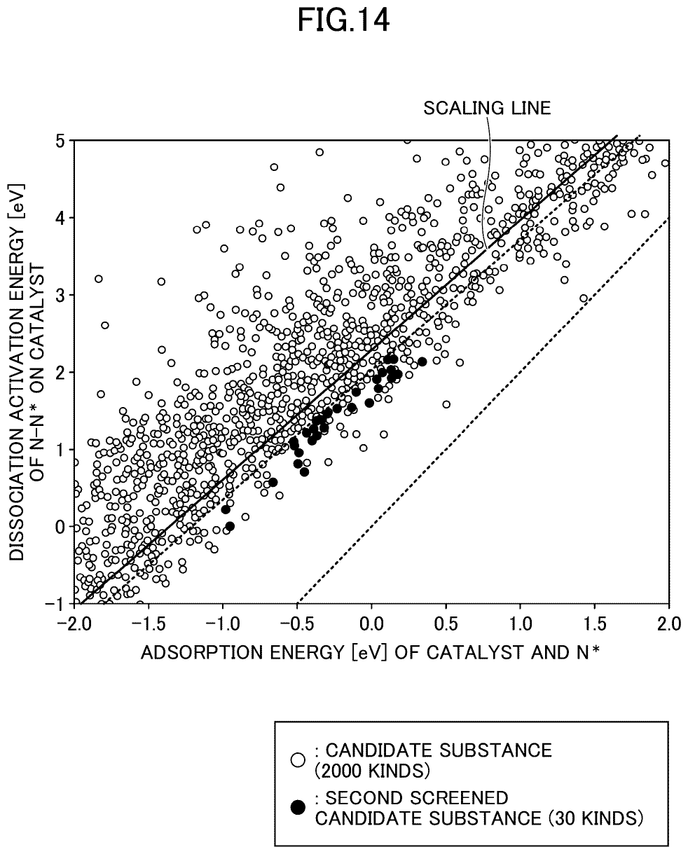

FIG. 14 illustrates an example of a result of plotting the descriptors of the candidate substances. Similar to FIG. 12, FIG. 14 illustrates an activity map illustrating the relationship between the adsorption energy (Ex) of the intermediate (N*) of nitrogen, and the dissociation activation energy (E_{N-N}) of the transition state (N-N*) of nitrogen on the catalyst, as the descriptors. In addition, the illustration of the yield is omitted in order to clearly illustrate the plotted points.

Moreover, the first screening process (step S16), when narrowing down the first screened candidate substances by plotting the candidate substances on the activity map by the first screening unit 16, the data table listing only the information related to the candidate substances 1, 2, 3, . . . , N (N is an integer greater than or equal to 1) may be used as described above (refer to FIG. 8). Further, the candidate substances in the data table listing only the candidate substances may be sorted in an order included in the high activity region, and the candidate substances included in the high activity region and greater than or equal to a threshold value may be narrowed down as the first screened candidate substances. The first screening unit 16 may narrow down the first screened candidate substances by plotting the candidate substances on the activity map by the first plotting unit 15, using the sorted data table.

Next, as illustrated in FIG. 11, the second calculation unit 17 calculates the descriptor related to the catalytic reaction with respect to the first screened candidate substances using the first screened candidate substances, in a state where the surface of the first screened candidate substances is relaxed (second calculation process: step S17).

The present embodiment calculates, as the descriptor, similar to the first energy, the second energy including the second intermediate energy of the intermediate of the reactant (N₂, H₂) used for the descriptor and adsorbed on the surface of the first screened candidate substance, and the second transition state energy of the dissociation reaction in which the reactant is separated into two or more substances after the reactant is adsorbed on the surface of the first screened candidate substance.

In the second calculation process (step S17), as in the first calculation process (step S14), the second calculation unit 17 preferable calculates the second energy of the first screened candidate substance, using the machine learning potential 30.

In the second calculation process (step S17), in the case where the descriptor is the second intermediate energy, the second calculation unit 17 preferably optimizes the structure of the intermediate structure including the first screened candidate substance and the reactant in the state where the surface of the first screened candidate substance is relaxed, similar to in the first calculation process (step S14). In this case, it possible to select the intermediate structure having a stable structure.

In the second calculation process (step S17), the second calculation unit 17 preferably extracts a structure in which the adsorption position of the reactant with respect to the first screened candidate substance is correct and the second intermediate energy of the first screened candidate substance and the intermediate of the reactant is most stable, similar to the first calculation process (step S14). In this case, it is possible to calculate the second intermediate energy of the reactant with a high accuracy.

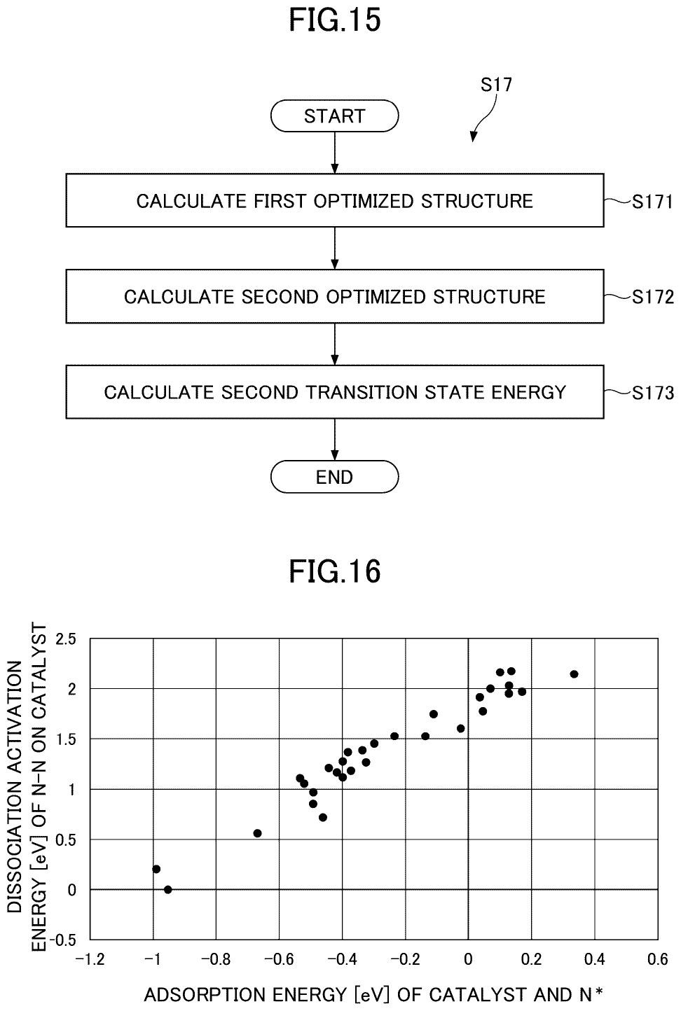

In addition, as illustrated in FIG. 15, the second calculation unit 17 preferably performs the first structure optimizing process (step S171), the second structure optimization process (step S172), and the second transition state energy acquisition process (step S173), similar to the first calculation process (step S14). In this case, it is possible to calculate the transition state energy of the transition state of the intermediate with a high accuracy.

In the first structure optimization process (step S171), the first structure optimization unit 171 optimizes the structure of the intermediate structure including the first screened candidate substance and the reactant on the surface of the first screened candidate substance included in the dissociation reaction from one molecule to two molecules, and calculates the first optimized structure. That is, in the first structure optimization process (step S171), the first structure optimization unit 171 optimizes the structure of the intermediate structure including the first screened candidate substance and the reactant in the state where the surface of the first screened candidate substance is relaxed in the dissociation reaction from one molecule to two molecules, and calculates the first optimized structure.

In the second structure optimization process (step S172), the second structure optimization unit 172 optimizes the separation structure of the reactant when the reactant is separated into two or more substances, and calculates the second optimized structure, similar to the second structure optimization unit 142.

In the second transition state energy acquisition process (step S173), the second transition state energy acquisition unit 173 calculates the second transition state energy, which is the transition state energy, from the first optimized structure obtained in the first structure optimization process (step S171) and the second optimized structure obtained in the second structure optimization process (step S172), similar to the first transition state energy acquisition unit 143.

For example, AB* is the molecule adsorbed on the candidate substance, A-B* is the molecule in the transition state of AB* in the process of separating AB* into A* and B*, A* is the intermediate of the molecule A, B* is the intermediate of the molecule B, as described above, and the reaction in which one molecule AB* separates into two molecules A* and B* is assumed to progress as in the reaction formula (1) described above.

As indicated by the formula (1) described above, in the case where the dissociation reaction in which AB* changes from one molecule to two molecules occurs, the first structure optimization process (step S171), arranges by the first structure optimization unit 171, AB* on the surface of the first screened candidate substance in the state where the surface of the first screened candidate substance is relaxed, and optimizes the structure of the intermediate structure including the first screened candidate substance and AB*, similar to the first structure optimization process (step S141).

The second structure optimization process (step S172) optimizes the separation structure so that AB* separates into two molecules A* and B*.

In the transition state energy acquisition process (step S173), the second transition state energy acquisition unit 173 calculates, as the second transition state energy, the transition state energy required for one molecule AB* to separate into two molecules A* and B*, similar to the transition state energy acquisition process (step S143).

Next, as illustrated in FIG. 11, the second plotting unit 18 plots the first screened candidate substances on the activity map (refer to FIG. 12) created by the map creation process (step S12) based on the second energy (the energy including the second intermediate energy and the second transition state energy) which is the descriptor calculated by the second calculation unit 17, and creates the second plot map (second plotting process: step S18).

Next, the second screening unit 19 screens the first screened candidate substances based on the second plot map created by the second plotting process (step S18), and selects the second screened candidate substances (second screening process: step S19).

That is, the second screening unit 19 can select, as the second screened candidate substances, the first screened candidate substances plotted in the high activity region including the catalyst having the high activity by the second plotting process (step S18).

For example, 30 kinds to 100 kinds of second screened candidate substances are selected. FIG. 16 illustrates an example of the activity map in which 30 kinds of second screened candidate substances are plotted, for example. The number of the second screened candidate substances to be selected can be appropriately selected according to the number of the first screened candidate substances or the like as described above, and may be several kinds or several tens of kinds, for example.

Next, the filtering unit 21 may narrow down the second screened candidate substances obtained in the second screening process (step S19), based on the catalyst stability or the catalyst cost (filtering process: step S20).

The second screened candidate substances are narrowed down to 30 kinds to 50 kinds, for example. The number of second screened candidate substances to be narrowed down to can be appropriately selected according to the number of second screened candidate substances to be selected or the like, and may be several kinds to several tens of kinds, for example.

Next, the second screened candidate substances selected by the second screening unit 19 or the second screened candidate substances narrowed down to by the filtering unit 21 are displayed or the like and output by the output unit 22 (output process: step S21).

The method for selecting the catalyst according to the present embodiment enables the selection of the NH₃ synthesis catalyst having a good NH₃ production efficiency.

Examples of the NH₃ synthesis catalyst having the good NH₃ production efficiency selected by the method for selecting the catalyst according to the present embodiment include IrSc, FePd₃, MnTc₃, IrY, CrPd₃, MnPd₃, RhY, Co₃Pt, CrPt₃, FeRh₃, CrRh₃, Ni₃Ti, Ir₃V, Pt₃Ti, Co₃Rh, Pd₃Ti, Ni₃Zr, Co₃W, NiPd₃, FeNi₃, Ir₃Mn, IrMn, MnPt, MnNi₃, Ir₃Re, MnRh, Pd₃V, MnPt₃, Rh₃V, Rh₃Ti, or the like.

Whether or not the catalyst selected by the method for selecting the catalyst according to the present embodiment is a NH: synthesis catalyst having a high yield and a good NH; production efficiency can be determined, for example, by comparing the energy of the catalyst selected by the method for selecting the catalyst according to the present embodiment with the energy of the catalyst selected by the conventional quantum chemistry calculation that is generally used, and verifying the deviation therefrom.

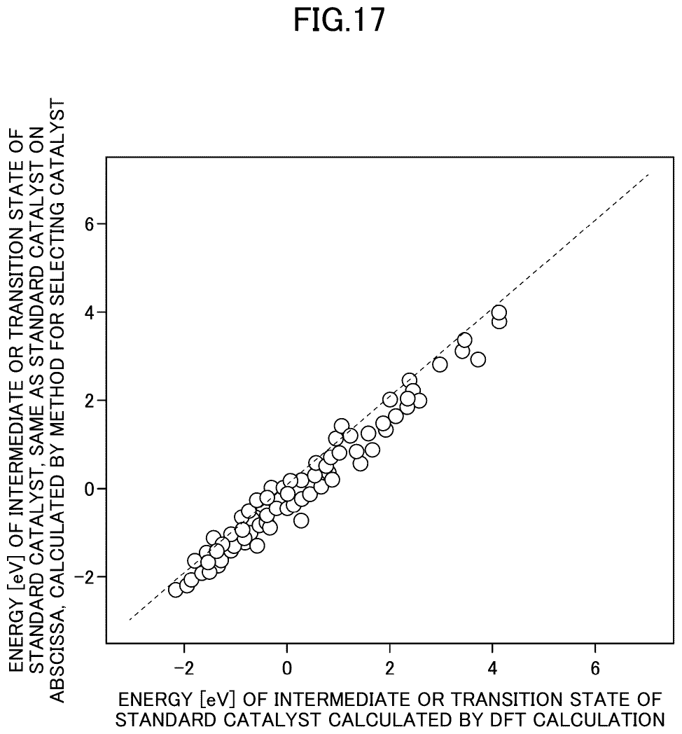

FIG. 17 is a diagram illustrating a correlation between the energy of the intermediate or the transition state of the standard catalyst calculated by the DFT calculation and the energy of the intermediate or the transition state of the standard catalyst, which is the same as the standard catalyst on the abscissa, calculated by the method for selecting the catalyst according to the embodiment of the present invention, which appear in the elementary reactions related to the NH₃ synthesis in the standard catalysts required when creating the activity map. The DFT calculation is a technique of the conventional quantum chemistry calculation that is generally used. The standard catalyst required when creating the activity map is an elemental metal, such as Co, Rh, Ru, Cu, Fe, Re, or the like, or the like. As illustrated in FIG. 17, because a linear relationship stands between the plotted catalysts, the machine learning potential 30 can reproduce substantially the same result as the calculation result obtained by the DFT calculation.

Hence, it may be regarded that the energies of the intermediate and the transition state of various catalysts can be predicted with high accuracy, by using the method for selecting the catalyst according to the present embodiment.

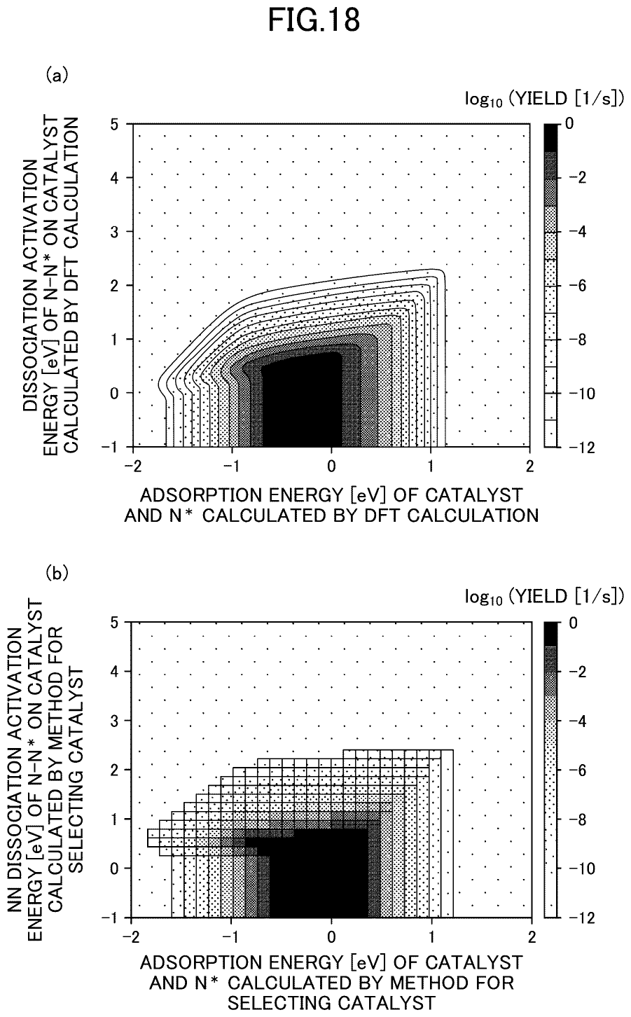

An example of the activity map of the catalyst created using the DFT calculation is illustrated in FIG. 18. FIG. 18 also illustrates the activity map of the catalyst created using the method for selecting the catalyst according to the present embodiment. As illustrated in FIG. 18, the activity map of the catalyst selected using the method for selecting the catalyst according to the present embodiment shows substantially the same tendency as the activity map of the catalyst created using the DFT calculation. Hence, the energy calculated by the machine learning potential 30 can be used to create and reproduce an activity map that is substantially similar to the activity map created using the energy calculated by the DFT calculation.

For this reason, it may be regarded that the catalyst selected by the method for selecting catalyst according to the present embodiment is a NH₃ synthesis catalyst having a good NH₃ production efficiency.

In the present embodiment, the method for selecting the catalyst according to the present embodiment may not include or omit the first calculation process (step S14), the first plotting process (step S15), and the first screening process (step S16), or the second calculation process (step S17), the second plotting process (step S18), and the second screening process (step S19), according to the number of candidate substances prepared in the preparation process (step S13). That is, in the method for selecting the catalyst according to the present embodiment, the candidate substances may be narrowed down by omitting the calculation in the state where all of the candidate substances are fixed, or omitting the calculation in the state where the surface of the candidate substance is relaxed.

As described above, the method for selecting the catalyst according to the present embodiment includes the descriptor selection process (step S11), the map creation process (step S12), the preparation process (step S13), the first calculation process (step S14), the first plotting process (step S15), the first screening process (step S16), the second calculation process (step S17), the second plotting process (step S18), and the second screening process (step S19).

In the method for selecting the catalyst according to the present embodiment, the first calculation process (step S14) calculates the first energy of the plurality of candidate substances prepared by the preparation process (step S13), and the first screening process (step S16) narrows down the first screened candidate substances from the candidate substances plotted on the first plot map created by the first plotting process (step S15). Further, in the method for selecting the catalyst according to the present embodiment, the second calculation process (step S17) calculates the second energy of the selected first screened candidate substances, and the second screening process (step S16) selects the second screened candidate substances from the first screened candidate substances plotted on the second plot map created by the second plotting process (step S18).

That is, first, in a first stage of the method for selecting the catalyst according to the present embodiment, the descriptors related to the catalytic reactions using the candidate substances are calculated in a short time, although with a low accuracy, in the state where all of candidate substances are fixed, the calculated descriptors are plotted on the activity map, and the first screened candidate substances are narrowed down from the plotted candidate substances. Next, in a second stage of the method for selecting the catalyst according to the present embodiment, the descriptors related to the catalytic reactions using the narrowed down first screened candidate substances are recalculated with a high accuracy in the state where the surface of the first screened candidate substances is relaxed. Then, the method for selecting the catalyst according to the present embodiment narrows down the first screened candidate substances further based on the calculated more accurate descriptors of the first screened candidate substances, and selects the second screened candidate substances. Hence, the method for selecting the catalyst according to the present embodiment can select an effective catalyst from candidate substances quickly and with a high accuracy, and can thus efficiently select the NH₃ synthesis catalyst to be used for the catalytic reaction for synthesizing the target product (NH₃) from the raw materials (H₂, N₂).

When synthesizing the target product, such as a compound or the like, various chemical reactions are used industrially, but there are cases where it is necessary to apply pressure, temperature, or the like during the reaction process, thereby requiring a large amount of energy. The method for selecting the catalyst according to the present embodiment can reduce the activation energy of the chemical reaction, and can thus be used effectively for selecting the catalyst which can reduce the energy required for the chemical reaction.

In particular, NH₃, which is the target product, is attracting attention as one of the promising candidates for hydrogen carrier, and it is important to use the NHs synthesis catalyst capable of efficiently synthesizing NH₃, in order to produce NH₃ with a good production efficiently. NH₃ is generally synthesized using the Haber-Bosch (HB) process, but because the process is performed at high temperature and high pressure, improvement of the NHs synthesis method is important from a viewpoint of reducing CO₂ emissions and improving energy efficiency. The method for selecting the catalyst according to the present embodiment can be suitably used as a selection method for searching for a synthesis catalyst having a good production efficiency of NH₃, which is the target product.

In the method for selecting the catalyst according to the present embodiment, the first calculation process (step S14) and the second calculation process (step S17) can use the machine learning potential 30 and calculate the descriptors related to the catalytic reactions using the first screened candidate substances. Hence, the method for selecting the catalyst according to the present embodiment can further reduce the calculation time of the descriptors, and can thus reduce a selection time of the NHa synthesis catalyst having a good production efficiency.

In the method for selecting the catalyst according to the present embodiment, the first calculation process (step S14) and the second calculation process (step S17) can vary the adsorption position of the reactant on the surface of the candidate substance or the first screened candidate substances, and optimize the structure of the intermediate structure including the candidate substance or the first screened candidate substance and the reactant. Hence, the method for selecting the catalyst according to the present embodiment can obtain a stable structure of the intermediate structure in which the reactant is adsorbed on the surface of the candidate substance or the first screened candidate substance, and appropriately calculate the first intermediate energy and the second intermediate energy with ease as the descriptors.

In the method for selecting the catalyst according to the present embodiment, the first calculation process (step S14) may include the first structure optimization process (step S141), the second structure optimization process (step S142), and the first transition state energy acquisition process (step S143). In the case where the first calculation process (step S14) includes these processes, after the transition state of the intermediate of the reactant is adsorbed on the surface of the candidate substance, the first transition state energy of the dissociation reaction in which the intermediate of the reactant is separated into two or more substances can be calculated as the descriptor. Hence, in the method for selecting the catalyst according to the present embodiment, the first calculation process (step S14) can appropriately calculate the NH₃ synthesis rate with ease for each type of catalyst, and can thus more appropriately select the NH₃ synthesis catalyst having the good production efficiency.

In the method for selecting the catalyst according to the present embodiment, the second calculation process (step S17) can include the first structure optimization process (step S171), the second structure optimization process (step S172), and the second transition state energy acquisition process (step S173). In the case where the second calculation process (step S17) includes these processes, after the transition state of the intermediate of the reactant is adsorbed on the surface of the first screened candidate substance, the second transition state energy of the dissociation reaction in which the intermediate of the reactant is separated into two or more substances can be calculated as the descriptor. Hence, in the method for selecting the catalyst according to the present embodiment, the second calculation process (step S17) can appropriately calculate the NH₃ synthesis rate with ease for each type of catalyst, and can thus more appropriately select the NH₃ synthesis catalyst having the good production efficiency.

The preparation process (step S12) of the method for selecting the catalyst according to the present embodiment may include, as another candidate substance, a substance obtained by substituting one kind of element on the surface of one candidate substance with another, different element. In this case, the method for selecting the catalyst according to the present embodiment can include a substance having a partially different structure as the candidate substance, and can thus select in more detail the NH₃ synthesis catalyst having the good NH₃ production efficiency.

In the method for selecting the catalyst according to the present embodiment, the preparation process (step S12) can include, as the candidate substances, substances in which only the crystal planes are different. Hence, the method for selecting the catalyst according to the present embodiment can prepare, as the surface of the candidate substances, a plurality of surfaces having different crystal planes. For this reason, the method for selecting the catalyst according to the present embodiment can use the candidate substance having a surface with a particularly high reaction rate among the plurality of surfaces of the candidate substance, and can thus appropriately improve the efficiency of NH₃ synthesis.

The method for selecting the catalyst according to the present embodiment can include the filtering process (step S18). Hence, the method for selecting the catalyst according to the present embodiment can further narrow down the second screened candidate substances obtained in the second screening process (step S16) by taking into consideration the catalyst stability, the catalyst cost, or the like, and can thus select the NH: synthesis catalyst having the good NH₃ production efficiency while stabilizing and reducing the cost of NH₃ synthesis.

In the method for selecting the catalyst according to the present embodiment, the adsorption energy of the reactant adsorbed on the surface of the candidate substance, and the transition state energy of the dissociation reaction in which the reactant is separated into two or more substances after the reactant is adsorbed on the surface of the candidate substance, can be used as the descriptors. That is, in the method for selecting the catalyst according to the present embodiment, the adsorption energy with which the intermediate N* of N₂ is adsorbed on the NH₃ synthesis catalyst can be used as the first intermediate energy and the second intermediate energy, and the dissociation activation energy at which the transition state N-N* of the intermediate of N₂ dissociates from the NH₃ synthesis catalyst can be used as the first transition state energy and the second transition state energy, as descriptors. In the process of synthesizing NH₃ from the raw materials N₂ and H₂, the adsorption energy (E_{N}) of N* on the catalyst, and the dissociation activation energy (E_{N-N}) of N-N* on the catalyst, greatly affect the NH₃ synthesis. The method for selecting the catalyst according to the present embodiment can appropriately determine the NH: synthesis rate with a high accuracy, using the activity map in which the adsorption energy (E_{N}) of the intermediate N* of nitrogen and the dissociation activation energy (E_{N-N}) of the transition state N-N* of nitrogen on the catalyst are used as the intermediate energy and the transition state energy, respectively, and can thus more appropriately select the NH₃ synthesis catalyst having the good production efficiency.

### <Catalyst>

The catalyst according to the present embodiment is selected by the method for selecting the catalyst according to the present embodiment.

The catalyst according to the present embodiment can be suitably used as the catalyst used for producing a target product from raw materials, such as the NH₃ synthesis catalyst.

In the case where the catalyst according to the present embodiment is used as the NHs synthesis catalyst, IrSc, FePd₃, MnTc₃, IrY, CrPd₃, MnPd₃, RhY, Co₃Pt, CrPt₃, FeRhe, CrRh₃, Ni₃Ti, Ir₃V, Pt₃Ti, Co₃Rh, Pd₃Ti, Ni₃Zr, Co₃W, NiPd₃, FeNi₃, Ir₃Mn, IrMn, MnPt, MnNi₃, Ir₃Re, MnRh, Pd₃V, MnPt₃, Rh₃V, Rh₃Ti, or the like may be used as the NH₃ synthesis catalyst, as described above. The catalyst may use one of these substances, or a combination of two or more of these substances.

The catalyst according to the present embodiment can be selected by the method for selecting the catalyst according to the present embodiment described above, and thus, the target product can be produced efficiently.

### <Method for Producing Catalyst>

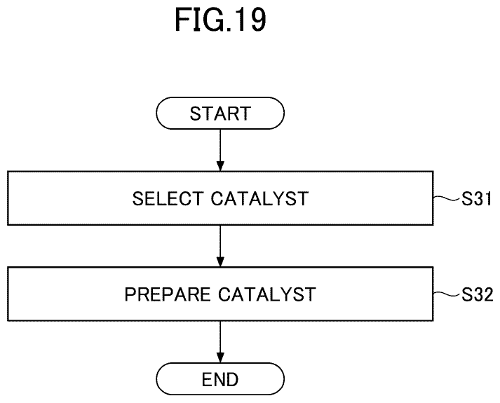

FIG. 19 is a flow chart illustrating an example of a method for producing the catalyst according to the present embodiment. As illustrated in FIG. 19, the method for producing the catalyst according to the present embodiment includes a selection process (step S31) for selecting the catalyst, and a preparation process (step S32) for preparing the catalyst.

In the selection process (step S31), the catalyst is selected by the method for selecting the catalyst according to the present embodiment described above.

In the preparation process (step S32), the catalyst selected by the selection process (step S31) is prepared. The method for preparing the catalyst is not particularly limited, and a general preparation method may be used depending on the type of the selected catalyst.

Because the method for producing the catalyst according to the present embodiment can prepare the catalyst selected by the method for selecting the catalyst according to the present embodiment described above, it is possible to appropriately produce a catalyst capable of efficiently producing the target product.

Although the embodiments are described above, the embodiments are presented as examples, and the present invention is not limited to these embodiments. The embodiments described above can be implemented in various other forms, and various combinations, omissions, substitutions, variations, or the like can be made without departing from the scope of the subject matte of the present invention. The embodiments and modifications thereof are included in the scope of the subject matter of the present invention, and are included in the present invention described in the claims and the scope of equivalents thereof.

Aspects of the embodiments of the present invention include the following, for example.

<1> A method for selecting a catalyst to be used for a catalytic reaction for producing a target product from raw materials, the method comprising the steps of:
selecting, as a descriptor, an energy of an intermediate structure or a transition state structure included in an elementary reaction of the catalytic reaction;
creating a map representing a relationship between the descriptor and a reactivity of the catalyst;
preparing a plurality of kinds of candidate substances for the catalyst;
first calculating the descriptor related to the catalytic reaction using the candidate substances in a state where the candidate substances are fixed;
first plotting the descriptor calculated by the first calculating step on the map and creating a first plot map;
first screening the candidate substances based on the first plot map and selecting first screened candidate substances;
second calculating the descriptor for the catalytic reaction using the first screened candidate substances in a state where a surface of the first screened candidate substances is relaxed;
second plotting the descriptor calculated by the second calculating step on the map and creating a second plot map; and
second screening the first screened candidate substances based on the second plot map and selecting second screened candidate substances.

<2> The method for selecting the catalyst according to <1>, wherein at least one of the first calculating step and the second calculating step calculates the descriptor using a machine learning potential.

<3> The method for selecting the catalyst according to <2>, wherein the machine learning potential is a neural network potential.

<4> The method for selecting the catalyst according to any one of <1> to <3>, wherein at least one of the first calculating step and the second calculating step varies an adsorption position of a reactant including a substance derived from the raw materials on the surface of the candidate substances included in the elementary reaction, and optimizes a structure of an intermediate structure including the candidate substances and the reactant.

<5> The method for selecting the catalyst according to any one of <1> to <4>, wherein:
the descriptor includes a transition state energy of a dissociation reaction in which a reactant including the substance derived from the raw materials is adsorbed on a surface of the catalyst and is thereafter separated into two or more substances, and
the first calculating step includes the steps of:
   optimizing a structure of an intermediate structure including the candidate substances and the reactant, on a surface of the candidate substances included in the dissociation reaction, and calculating a first optimized structure;
   optimizing a structure including candidate substances and the reactant when the reactant is separated into the two or more substances, and
   calculating a second optimized structure; and calculating a first transition state energy, which is the transition state energy, from the first optimized structure and the second optimized structure.

<6> The method for selecting the catalyst according to any one of <1> to <5>, wherein:
the descriptor includes a transition state energy of a dissociation reaction in which a reactant including a substance derived from the raw materials is adsorbed on a surface of the catalyst and the reactant is thereafter separated into two or more substances, and
the second calculation step includes the steps of:
   optimizing a structure of an intermediate structure including the first screened candidate substances and the reactant, on a surface of the first screened candidate substances included in the dissociation reaction, and calculating a first optimized structure;
   optimizing a separation structure of the reactant when the reactant is separated into the two or more substances, and calculating a second optimized structure; and
   calculating a second transition state energy, which is the transition state energy, from the first optimized structure and the second optimized structure.

<7> The method for selecting the catalyst according to any one of <1> to <6>, wherein the preparing step includes, as another candidate substance, a substance obtained by substituting one kind of element on the surface of one candidate substance with another, different element.

<8> The method for selecting the catalyst according to any one of <1> to <7>, wherein the preparing step includes, as the candidate substances, substances in which only the crystal planes are different.

<9> The method for selecting the catalyst according to any one of <1> to <8>, further comprising the step of:
narrowing down the second screened candidate substances obtained by the second screening step by taking into consideration a catalyst stability or a catalyst cost.

<10> The method for selecting the catalyst according to any one of <1> to <9>, wherein the candidate substances are a single metal, an alloy including a plurality of metals, or a metal compound including a metal.

<11> The method for selecting the catalyst according to any one of <1> to <10>, wherein the raw materials are nitrogen and hydrogen, and the target product is ammonia.

<12> The method for selecting the catalyst according to <11>, wherein the descriptor includes an adsorption energy when a reactant including the substance derived from the raw materials is adsorbed on the surface of the candidate substances, and a transition state energy of a dissociation reaction in which the reactant is separated into two or more substances after the reactant is adsorbed on the surface of the candidate substances.

<13> A catalyst selected by the method for selecting the catalyst according to any one of <1> to <12>.

<14> The catalyst according to <13>, which is a catalyst for ammonia synthesis, and includes one or more kinds of components selected from a group consisting of IrSc, FePd₃, MnTc₃, IrY, CrPd₃, MnPd₃, RhY, Co₃Pt, CrPt₃, FeRh₃, CrRh₃, Ni₃Ti, Ir₃V, Pt₃Ti, CO₃Rh, Pd₃Ti, Ni₃Zr, Co₃W, NiPd₃, FeNi₃, Ir₃Mn, IrMn, MnPt, MnNi₃, Ir₃Re, MnRh, Pd₃V, MnPt₃, RheV, and Rh₃Ti.

<15> A method for producing a catalyst, comprising the steps of:
selecting the catalyst selected by the method for selecting the catalyst according to any one of <1> to <12>; and
preparing the catalyst selected by the catalyst selecting step.

This application is based upon and claims priority to Japanese Patent Application No. 2022-107477, filed on Jul. 4, 2022 before the Japan Patent Office, the entire contents of which are incorporated herein by reference.

### DESCRIPTION OF REFERENCE NUMERALS

1: Catalyst selection system
10: Catalyst selection device
11: Descriptor selection unit
12: Map creation unit
13: Preparation unit
14: First calculation unit
15: First plotting unit
16: First screening unit
17: Second calculation unit
18: Second plotting unit
19: Second screening unit
21: Filtering unit
22: Output unit
20: Storage device
30: Machine learning potential
141, 171: First structure optimization unit
142, 172: Second structure optimization unit
143: First transition state energy acquisition unit
173: Second transition state energy acquisition unit

1. A method for selecting a catalyst to be used for a catalytic reaction for producing a target product from raw materials, the method comprising:
selecting, as a descriptor, an energy of an intermediate structure or a transition state structure included in an elementary reaction of the catalytic reaction;
creating a map representing a relationship between the descriptor and a reactivity of the catalyst;
preparing a plurality of kinds of candidate substances for the catalyst;
first calculating the descriptor related to the catalytic reaction using the candidate substances in a state where the candidate substances are fixed;
first plotting the descriptor calculated by the first calculating on the map and creating a first plot map;
first screening the candidate substances based on the first plot map and selecting first screened candidate substances;
second calculating the descriptor for the catalytic reaction using the first screened candidate substances in a state where a surface of the first screened candidate substances is relaxed;
second plotting the descriptor calculated by the second calculating on the map and creating a second plot map; and
second screening the first screened candidate substances based on the second plot map and selecting second screened candidate substances.

2. The method for selecting the catalyst as claimed in claim 1, wherein at least one of the first calculating and the second calculating calculates the descriptor using a machine learning potential.

3. The method for selecting the catalyst as claimed in claim 2, wherein the machine learning potential is a neural network potential.

4. The method for selecting the catalyst as claimed in claim 1, wherein at least one of the first calculating and the second calculating varies an adsorption position of a reactant including a substance derived from the raw materials on the surface of the candidate substances included in the elementary reaction, and optimizes a structure of an intermediate structure including the candidate substances and the reactant.

5. The method for selecting the catalyst as claimed in claim 1, wherein:
the descriptor includes a transition state energy of a dissociation reaction in which a reactant including the substance derived from the raw materials is adsorbed on a surface of the catalyst and is thereafter separated into two or more substances, and
the first calculating includes:
optimizing a structure of an intermediate structure including the candidate substances and the reactant, on a surface of the candidate substances included in the dissociation reaction, and calculating a first optimized structure;
optimizing a structure including candidate substances and the reactant when the reactant is separated into the two or more substances, and calculating a second optimized structure; and
calculating a first transition state energy, which is the transition state energy, from the first optimized structure and the second optimized structure.

6. The method for selecting the catalyst as claimed in claim 1, wherein:
the descriptor includes a transition state energy of a dissociation reaction in which a reactant including a substance derived from the raw materials is adsorbed on a surface of the catalyst and the reactant is thereafter separated into two or more substances, and
the second calculation includes:
optimizing a structure of an intermediate structure including the first screened candidate substances and the reactant, on a surface of the first screened candidate substances included in the dissociation reaction, and calculating a first optimized structure;
optimizing a separation structure of the reactant when the reactant is separated into the two or more substances, and calculating a second optimized structure; and
calculating a second transition state energy, which is the transition state energy, from the first optimized structure and the second optimized structure.

7. The method for selecting the catalyst as claimed in claim 1, wherein the preparing includes, as another candidate substance, a substance obtained by substituting one kind of element on the surface of one candidate substance with another, different element.

8. The method for selecting the catalyst as claimed in claim 1, wherein the preparing includes, as the candidate substances, substances in which only the crystal planes are different.

9. The method for selecting the catalyst as claimed in claim 1, further comprising:
narrowing down the second screened candidate substances obtained by the second screening by taking into consideration a catalyst stability or a catalyst cost.

10. The method for selecting the catalyst as claimed in claim 1, wherein the candidate substances are a single metal, an alloy including a plurality of metals, or a metal compound including a metal.

11. The method for selecting the catalyst as claimed in claim 1, wherein the raw materials are nitrogen and hydrogen, and the target product is ammonia.

12. The method for selecting the catalyst as claimed in claim 11, wherein the descriptor includes an adsorption energy when a reactant including the substance derived from the raw materials is adsorbed on the surface of the candidate substances, and a transition state energy of a dissociation reaction in which the reactant is separated into two or more substances after the reactant is adsorbed on the surface of the candidate substances.

13. A catalyst selected by the method for selecting the catalyst according to claim 1.

14. The catalyst as claimed in claim 13, which is a catalyst for ammonia synthesis, and includes one or more kinds of components selected from a group consisting of IrSc, FePd₃, MnTc₃, IrY, CrPd₃, MnPd₃, RhY, Co₃Pt, CrPt₃, FeRh₃, CrRh₃, Ni₃Ti, Ir₃V, Pt₃Ti, Co₃Rh, Pd₃Ti, Ni₃Zr, Co₃W, NiPd₃, FeNi₃, Ir₃Mn, IrMn, MnPt, MnNi₃, Ir₃Re, MnRh, Pd₃V, MnPt₃, Rh₃V, and Rh₃Ti.

15. A method for producing a catalyst, comprising:
selecting the catalyst selected by the method for selecting the catalyst according to claim 1; and
preparing the catalyst selected by the catalyst selecting.

16. A computer-implemented method for selecting a catalyst to be used for a catalytic reaction for producing a target product from raw materials, the method comprising:
selecting, as a descriptor, an energy of an intermediate structure or a transition state structure included in an elementary reaction of the catalytic reaction;
creating a map representing a relationship between the descriptor and a reactivity of the catalyst;
preparing a plurality of kinds of candidate substances for the catalyst;
first calculating the descriptor related to the catalytic reaction using the candidate substances in a state where the candidate substances are fixed;
plotting the descriptor calculated by the first calculating on the map to create a first plot map;
screening the candidate substances based on the first plot map to select first screened candidate substances;
second calculating the descriptor for the catalytic reaction using the first screened candidate substances in a state where a surface of the first screened candidate substances is relaxed;
plotting the descriptor calculated by the second calculating on the map to create a second plot map;
screening the first screened candidate substances based on the second plot map to select second screened candidate substances; and
outputting the second screened candidate substances to a display.

17. A catalyst for ammonia synthesis comprising:
the catalyst selected by the computer-implemented method for selecting the catalyst according to claim 16,
wherein the catalyst includes one or more kinds of components selected from a group consisting of IrSc, FePd₃, MnTc₃, IrY, CrPd₃, MnPd₃, RhY, Co₃Pt, CrPt₃, FeRh₃, CrRh₃, Ni₃Ti, Ir₃V, Pt₃Ti, Co₃Rh, Pd₃Ti, Ni₃Zr, Co₃W, NiPd₃, FeNi₃, Ir₃Mn, IrMn, MnPt, MnNi₃, Ir₃Re, MnRh, Pd₃V, MnPt₃, Rh₃V, and Rh₃Ti.

18. A method for producing a catalyst, comprising:
selecting the catalyst by the computer-implemented method for selecting the catalyst according to claim 16; and
preparing the selected catalyst.

19. The method for producing the catalyst as claimed in claim 18, wherein the selected catalyst includes one or more kinds of components selected from a group consisting of IrSc, FePd₃, MnTc₃, IrY, CrPd₃, MnPd₃, RhY, Co₃Pt, CrPt₃, FeRh₃, CrRh₃, Ni₃Ti, Ir₃V, Pt₃Ti, Co₃Rh, Pd₃Ti, Ni₃Zr, Co₃W, NiPd₃, FeNi₃, Ir₃Mn, IrMn, MnPt, MnNi₃, Ir₃Re, MnRh, Pd₃V, MnPt₃, Rh₃V, and Rh₃Ti.
